(19) United States
(12) Patent Application Publication
(10) Pub. No.: US 2023/0399660 A1
(43) Pub. Date: Dec. 14, 2023

(54) Cell Permeable Proteins for Genome Engineering

(71) Applicant: Altius Institute for Biomedical Sciences, Seattle, WA (US)

(72) Inventors: Alexander J. Federation, Renton, WA (US); Kyle Siebenthall, San Francisco, CA (US); Aidan Quigley, Philadelphia, PA (US); Alister PW Funnell, Seattle, WA (US); John A. Stamatoyannopoulos, Seattle, WA (US)

(21) Appl. No.: 18/033,000

(22) PCT Filed: Oct. 22, 2021
(86) PCT No.: PCT/US2021/056174

§ 371 Date: Apr. 20, 2023

Related U.S. Application Data
(60) Provisional application No. 63/105,007, filed on Oct. 23, 2020.

(51) Int. Cl. C12N 15/90; C12N 9/22; C07K 14/00

(52) U.S. Cl. CPC C12N 15/907; C12N 9/22; C07K 14/001; C12N 2800/80; C07K 2319/80

(57) ABSTRACT

The present disclosure provides genome engineering proteins, e.g., nucleic acid binding domains and/or functional domains that have a net positive charge and are cell permeable and can be introduced into the cells without the use of a carrier such as micelles, vesicles, liposomes, and the like.

### CROSS-REFERENCE TO RELATED APPLICATIONS

This application claims priority to U.S. Provisional Application Ser. No. 63/105,007 filed Oct. 23, 2020, the disclosure of which is herein incorporated by reference in its entirety.

### INCORPORATION BY REFERENCE OF SEQUENCE LISTING PROVIDED AS A TEXT FILE

A Sequence Listing is provided herewith as a text file, “ALTI-731WO Seq List_ST25.txt,” created on Oct. 20, 2021 and having a size of 116,000 bytes. The contents of the text file are incorporated by reference herein in their entirety.

### INTRODUCTION

Genome engineering involves genome editing and gene regulation techniques which use nucleic acid binding domains that bind to a target nucleic acid. The nucleic acid binding domains are associated with (e.g., via fusion or interaction) functional domains that mediate genome editing or gene regulation. Nucleic acid binding domains and functional domains, if provided separately, can be introduced into cells as nucleic acids or proteins.

Introduction of proteins for genome engineering offers many advantages over introduction of nucleic acids. However, introduction of proteins into cells requires use of micelles, liposomes and other vehicles to transport the proteins across the cell membrane. Therefore, there is a need for cell permeable genome engineering proteins.

### SUMMARY

The present disclosure provides genome engineering proteins, e.g., nucleic acid binding domains and/or functional domains, that are cell permeable and can be introduced into the cells without the use of a carrier such as micelles, vesicles, liposomes, and the like. These proteins can include a nuclear localization sequence to facilitate movement into the nucleus where the genome engineering proteins can interact with a target gene.

In certain aspects, the genome engineering proteins have an overall positive charge. In certain embodiments, the genome engineering protein is a polypeptide comprising nucleic acid binding domains (NBD, e.g., DNA binding domain, DBD) that include repeat units (RUs) that mediate binding to a base in a nucleic acid. The RUs have been modified by substituting neutral or negatively charged amino acids with positively charged amino acids to render an overall positive charge to the RUs. These RUs are not naturally occurring RUs which may have a net positive charge.

In certain aspects, instead of or in addition to modifying the amino acid sequence of a genome engineering protein, a fusion partner is conjugated to the genome engineering protein, which fusion partner has an overall positive charge thereby rendering the conjugated genome engineering protein cell permeable.

### BRIEF DESCRIPTION OF THE DRAWINGS

FIG. 1. NBD comprising positively charged RUs conjugated to a positively charged first member of a heterodimer pair and KRAB conjugated to a positively charged second member of the heterodimer pair are transported across cell membrane and targeted to bind the TIM3 gene promoter, repressing TIM3 expression in a dose-dependent manner. Increasing amounts of the NBD decreases TIM3 expression.

### DETAILED DESCRIPTION

The present disclosure provides cell permeable genome engineering proteins that can be introduced into the cells without the use of a carrier such as micelles, vesicles, liposomes, and the like.

In certain aspects, the genome engineering proteins have been rendered cell permeable by modifying their amino acid sequence such that the proteins have an overall positive charge.

In certain aspects, instead of or in addition to modifying the amino acid sequence of a genome engineering protein, a fusion partner is conjugated to the genome engineering protein, which fusion partner has an overall positive charge thereby rendering the conjugated genome engineering protein cell permeable.

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a protein” includes a plurality of such proteins and reference to “the polynucleotide” includes reference to one or more polynucleotides, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflicts with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

As used herein, the term “derived” in the context of a polypeptide refers to a polypeptide that has a sequence that is based on that of a protein from a particular source (e.g., an animal pathogen such as Legionella). A polypeptide derived from a protein from a particular source may be a variant of the protein from the particular source (e.g., an animal pathogen such as Legionella). For example, a polypeptide derived from a protein from a particular source may have a sequence that is modified with respect to the protein's sequence from which it is derived. A polypeptide derived from a protein from a particular source shares at least 30% sequence identity with, at least 40% sequence identity with, at least 50% sequence identity with, at least 60% sequence identity with, at least 70% sequence identity with, at least 80% sequence identity with, or at least 90% sequence identity with the protein from which it is derived.

The term “modular” as used herein in the context of a nucleic acid binding domain, e.g., a modular animal pathogen derived nucleic acid binding domain (MAP-NBD) indicates that the plurality of repeat units present in the NBD can be rearranged and/or replaced with other repeat units and can be arranged in an order such that the NBD binds to the target nucleic acid. For example, any repeat unit in a modular nucleic acid binding domain can be switched with a different repeat unit. In some embodiments, modularity of the nucleic acid binding domains disclosed herein allows for switching the target nucleic acid base for a particular repeat unit by simply switching it out for another repeat unit. In some embodiments, modularity of the nucleic acid binding domains disclosed herein allows for swapping out a particular repeat unit for another repeat unit to increase the affinity of the repeat unit for a particular target nucleic acid. Overall, the modular nature of the nucleic acid binding domains disclosed herein enables the development of genome editing complexes that can precisely target any nucleic acid sequence of interest.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence, fusion proteins with heterologous and homologous leader sequences, with or without N-terminus methionine residues; immunologically tagged proteins; and the like. In specific embodiments, the terms refer to a polymeric form of amino acids of any length which include genetically coded amino acids. In particular embodiments, the terms refer to a polymeric form of amino acids of any length which include genetically coded amino acids fused to a heterologous amino acid sequence.

The term “heterologous” refers to two components that are defined by structures derived from different sources. For example, in the context of a polypeptide, a “heterologous” polypeptide may include operably linked amino acid sequences that are derived from different polypeptides (e.g., a NBD and a functional domain derived from different sources). Similarly, in the context of a polynucleotide encoding a chimeric polypeptide, a “heterologous” polynucleotide may include operably linked nucleic acid sequences that can be derived from different genes. Other exemplary “heterologous” nucleic acids include expression constructs in which a nucleic acid comprising a coding sequence is operably linked to a regulatory element (e.g., a promoter) that is from a genetic origin different from that of the coding sequence (e.g., to provide for expression in a host cell of interest, which may be of different genetic origin than the promoter, the coding sequence or both). In the context of recombinant cells, “heterologous” can refer to the presence of a nucleic acid (or gene product, such as a polypeptide) that is of a different genetic origin than the host cell in which it is present.

The term “operably linked” refers to linkage between molecules to provide a desired function. For example, “operably linked” in the context of nucleic acids refers to a functional linkage between nucleic acid sequences. By way of example, a nucleic acid expression control sequence (such as a promoter, signal sequence, or array of transcription factor binding sites) may be operably linked to a second polynucleotide, wherein the expression control sequence affects transcription and/or translation of the second polynucleotide. In the context of a polypeptide, “operably linked” refers to a functional linkage between amino acid sequences (e.g., different domains) to provide for a described activity of the polypeptide.

As used herein, the term “cleavage” refers to the breakage of the covalent backbone of a nucleic acid, e.g., a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, the polypeptides provided herein are used for targeted double-stranded DNA cleavage.

A “cleavage half-domain” is a polypeptide sequence which, in conjunction with a second polypeptide (either identical or different) forms a complex having cleavage activity (preferably double-strand cleavage activity).

A “target nucleic acid,” “target sequence,” or “target site” is a nucleic acid sequence that defines a portion of a nucleic acid to which a binding molecule, such as, the NBD disclosed herein will bind. The target nucleic acid may be present in an isolated form or inside a cell. A target nucleic acid may be present in a region of interest. A “region of interest” may be any region of cellular chromatin, such as, for example, a gene or a non-coding sequence within or adjacent to a gene, in which it is desirable to bind an exogenous molecule. Binding can be for the purposes of targeted DNA cleavage and/or targeted recombination, targeted activated or repression. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, promoter sequences, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

An “exogenous” molecule is a molecule that is not normally present in a cell but can be introduced into a cell by one or more genetic, biochemical or other methods. An exogenous molecule can comprise, for example, a functioning version of a malfunctioning endogenous molecule, e.g. a gene or a gene segment lacking a mutation present in the endogenous gene. An exogenous nucleic acid can be present in an infecting viral genome, a plasmid or episome introduced into a cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control region.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA, shRNA, RNAi, miRNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

“Modulation” of gene expression refers to a change in the activity of a gene. Modulation of expression can include, but is not limited to, gene activation and gene repression. Genome editing (e.g., cleavage, alteration, inactivation, donor integration, random mutation) can be used to modulate expression. Gene inactivation refers to any reduction in gene expression as compared to a cell that does not include a polypeptide or has not been modified by a polypeptide as described herein. Thus, gene inactivation may be partial or complete.

The terms “patient” or “subject” are used interchangeably to refer to a human or a non-human animal (e.g., a mammal).

The terms “treat”, “treating”, treatment” and the like refer to a course of action (such as administering a polypeptide comprising a NBD fused to a heterologous functional domain or a nucleic acid encoding the polypeptide) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject.

The terms “prevent”, “preventing”, “prevention” and the like refer to a course of action (such as administering a polypeptide comprising a NBD fused to a heterologous functional domain or a nucleic acid encoding the polypeptide) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The phrase “therapeutically effective amount” refers to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to a patient. The therapeutically effective amount can be ascertained by measuring relevant physiological effects.

The terms “conjugating,” “conjugated,” and “conjugation” refer to an association of two entities, for example, of two molecules such as two proteins, two domains (e.g., a binding domain and a cleavage domain), or a protein and an agent, e.g., a protein binding domain and a small molecule. The association can be, for example, via a direct or indirect (e.g., via a linker) covalent linkage or via non-covalent interactions. In some embodiments, the association is covalent. In some embodiments, two molecules are conjugated via a linker connecting both molecules. For example, in some embodiments where two proteins are conjugated to each other, e.g., a binding domain and a cleavage domain of an engineered nuclease, to form a protein fusion, the two proteins may be conjugated via a polypeptide linker, e.g., an amino acid sequence connecting the C-terminus of one protein to the N-terminus of the other protein. Such conjugated proteins may be expressed as a fusion protein.

The term “consensus sequence,” as used herein in the context of nucleic acid or amino acid sequences, refers to a sequence representing the most frequent nucleotide/amino acid residues found at each position in a plurality of similar sequences. Typically, a consensus sequence is determined by sequence alignment in which similar sequences are compared to each other. A consensus sequence of a protein can provide guidance as to which residues can be substituted without significantly affecting the function of the protein.

As used herein, the term “genome modifying proteins” refer to nucleic acid binding domains and functional domains which cooperate to modify genome or epigenome is a cell. Examples of genome modifying proteins are provided herein and include but are not limited to nucleic acid binding proteins comprising modular repeat units, nucleic acid binding proteins comprising zinc fingers, functional domains such as labels, tags, polypeptides having nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, e.g., nucleases, transcriptional activators, transcriptional repressors, chromatin modifying protein, and the like. Genome modifying proteins also encompass a single polypeptide comprising a nucleic acid binding domain and functional domain or two or more polypeptides, where a first polypeptide comprises a nucleic acid binding domain and a second polypeptide comprises a functional domain and wherein the first and second polypeptide associate with each other via a non-covalent interaction, such as, via a interactions mediated by first and second members of a heterodimer, where one of the first and second polypeptide is conjugated to the first member and the other polypeptide is conjugated to the second member. Such heterodimers are provided herein.

As used herein the terms “overall charge” or “net charge” refers to the theoretical charge of a protein at physiological pH based upon its amino acid sequence. In certain aspects, the amino acid substitutions disclosed herein may increase the theoretical net charge (at physiological pH) of the polypeptide being modified by at least +1, +2, +3, +4, +5, +10, +15, or more. In certain examples, a polypeptide of the present disclosure may have a net positive charge and may have a charge that is at least +1, +2, +3, +4, +5, +10, +15, or more than the net charge of the parent sequence from which the polypeptide is derived. For example, prior to a substitution, e.g., with a positively charged amino acid, a parent polypeptide may have a net charge of 0 and after a substitution the net charge is +1 or prior to a substitution, a parent polypeptide may have a net charge of +1 and after a substitution the net charge is +2 or more, and so on.

As used herein, a “fusion protein” includes a first protein moiety, e.g., a nucleic acid binding domain, having a peptide linkage with a second protein moiety. In certain aspects, the fusion protein is encoded by a single fusion gene. The first and second protein moieties may be linked directly, e.g., without intervening amino acids or may be linked via one or more amino acids, e.g., by a linker sequence.

### Positively Charged Genome and Epigenome Modifying Proteins

As set forth above, genome engineering proteins that are cell permeable and can be introduced into the cells without the use of a carrier such as micelles, vesicles, liposomes, and the like are disclosed herein. The genome engineering proteins have been rendered cell permeable by making the proteins positively charged as described below.

### Positively Charged Nucleic Acid Binding Domains

The present disclosure provides a genome engineering protein that may be a polypeptide comprising a nucleic acid binding domain (NBD, e.g., a DBD) comprising at least three repeat units (RUs) each comprising a 33-36 amino acid long sequence having at least 80% sequence identity to the amino acid sequence:

LTPDQ VVAIA SX¹²X¹³GG KQALE TVQRL LPVLC QDHG (SEQ ID NO:1), or

having the sequence of SEQ ID NO:1 with one or more conservative amino acid substitutions thereto; and comprising one or both of the following amino acid substitutions relative to SEQ ID NO:1: E20R/K/H and Q31K/R/H, wherein X¹² is any amino acid and X¹³ is any amino acid or absent,

wherein when the RUs comprise the substitution Q31K/R/H, X¹²X¹³ is not NK, YK or HN, the amino acid at position 32 is not P, the RUs further comprise the substitution E20R/K/H, and/or the RUs are 33-34 amino acid long; and

wherein when the RUs comprise the substitution E20R/K/H, X¹²X¹³ is not HD, HN, KG, KI, or the amino acid at position 32 is not P, the RUs further comprise the substitution Q31K/R/H, and/or the RUs are 33-34 amino acid long.

In certain embodiments, the RUs comprise the substitution Q31K/R/H and X¹²X¹³ is not NK, YK or HN. In certain embodiments, the RUs comprise the substitution Q31K/R/H and the amino acid at position 32 is not P and the RUs are 33-34 amino acid long. In certain embodiments, the RUs comprise the substitution E20R/K/H and the RUs are 33-34 amino acid long.

In certain embodiments, the RUs comprise the substitution E20R/K/H and X¹²X¹³ is not HD, HN, KG, or KI. In certain embodiments, the RUs comprise the substitution E20R/K/H and the amino acid at position 32 is not P. In certain embodiments, the RUs comprise the substitution E20R/K/H and the RUs further comprise the substitution Q31K/R/H. In certain embodiments, the RUs comprise the substitution E20R/K/H and the RUs are 33-34 amino acid long.

In certain embodiments, the RUs comprise the substitutions Q31K/R/H and E20R/K/H, e.g., the RUs comprise the substitutions Q31K and E20R or Q31K and E20K or Q31R and E20R.

In certain embodiments, the at least three RUs each comprise a 33-36 amino acid long sequence having at least 90% sequence identity to the amino acid sequence of SEQ ID NO:1. X₁₂X₁₃ is HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means X₁₃ is absent,

In certain embodiments, the at least three RUs comprise the amino acid sequence:

(SEQ ID NO: 158)
LTPDQ VVAIA S X¹²X¹³GG KQALR/K/H TVQRL LPVLC QDHG;
(SEQ ID NO: 159)
LTPDQ VVAIA S X¹²X¹³GG KQALE TVQRL LPVLC K/R/HDHG;
(SEQ ID NO: 160)
LTPDQ VVAIA S X¹²X¹³GG KQALR/K/H TVQRL LPVLC
K/R/HDHG;
or
(SEQ ID NO: 161)
LTPDQ VVAIA S X¹²X¹³GG KQALR TVQRL LPVLC KDHG.

In certain embodiments, the RUs as disclosed herein do not include one or more of the following substitutions: D4K/R/H, S11K/R/H; Q23K/R/H; C30K/R/H; and D32K/R/H.

In certain embodiments, the repeat units each have a theoretical net charge of at least +1 at physiological pH.

In certain embodiments, in addition to the indicated substitutions, the RU may comprise additional substitutions as compared to SEQ ID NO:1. For example, the additional substitutions may be up to 1, up to 2, up to 3, up to 4, up to 5, up to 6, up to 7, up to 8, up to 9, or up to 10 conservative amino acid substitutions as compared to SEQ ID NO:1.

In certain embodiments, the RU may comprise a 33-36 amino acid long sequence having a sequence at least 85%, at least 90%, at least 91%, at least 92%, at least 93%, at least 94%, at least 95%, or more identical to SEQ ID NO:1 and may further comprise one or more of the substitutions that increase the overall positive charge of the repeat unit.

In certain embodiments, the 33-36 long amino acid sequence of the repeat units does not comprise the amino acid sequence:

i.
(SEQ ID NO: 17)
LTPKQ VVAIA SX¹2X¹³GG KQALE TVQRL LPVLC QDHG
ii.
(SEQ ID NO: 18)
LTPRQ VVAIA SX¹²X¹³GG KQALE TVQRL LPVLC QDHG
iii.
(SEQ ID NO: 19)
LTPDQ VVAIA KX¹²X¹³GG KQALE TVQRL LPVLC QDHG
iv.
(SEQ ID NO: 20)
LTPDQ VVAIA RX¹²X¹³GG KQALE TVQRL LPVLC QDHG
v.
(SEQ ID NO: 21)
LTPDQ VVAIA SX¹²X¹³GG KQALE TVKRL LPVLC QDHG
vi.
(SEQ ID NO: 22)
LTPDQ VVAIA SX¹²X¹³GG KQALE TVRRL LPVLC QDHG
vii.
(SEQ ID NO: 23)
LTPDQ VVAIA SX¹²X¹³GG KQALE TVQRL LPVLK QDHG
viii.
(SEQ ID NO: 24)
LTPDQ VVAIA SX¹²X¹³GG KQALE TVQRL LPVLR QDHG
ix.
(SEQ ID NO: 25)
LTPDQ VVAIA SX₁₂X₁₃GG KQALE TVQRL LPVLC QKHG;
or
x.
(SEQ ID NO: 26)
LTPDQ VVAIA SX₁₂X₁₃GG KQALE TVQRL LPVLC QRHG,

wherein at least one of the amino acid residues at positions 4, 11, 23, and 32 has a positively charged side chain.

As noted herein, X¹² is any amino acid and X¹³ is any amino acid or absent. X¹²X¹³ may be a repeat variable diresidue (RVD), where the RVDs for individual RUs that can be selected to match the target nucleic acid sequence which the NBD is designed to bind. For example, the RVDs may be the RVDs present in TALEN proteins found in nature. For example, the RVDs X¹²X¹³ are selected from the group consisting of HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, and S*, where (*) means X¹³ is absent. The RVDs may be any of the expanded set of RVDs, including the non-canonical RVDs described in Miller et al., Nature Methods, Vol. 12, No. 5, May 2015. For example, the amino acid at the 12th position (X¹²) may be any one of amino acids G, A, S, V, T, C, I, L, N, D, Q, K, E, M, H, F, R, Y, or W, and the amino acid at the 13th position (X¹³) may be any one of amino acids G, A, S, P, V, T, I, N, D, K, or H, respectively, or absent. X¹²X¹³ may be selected from the group consisting of HG, VG, IG, EG, MG, YG, AA, EP, VA, QG, KG, RG, GN, SN, VN, LN, DN, QN, EN, HN, RH, NK, AN, FN, CI, HI, KI, RD, KD, ND, and AD. X¹²X¹³ may be selected from the group consisting of HG, VG, IG, EG, MG, YG, AA, EP, VA, QG, KG, RG, GN, VN, LN, DN, QN, EN, RH, NK, AN, FN, CI, HI, KI, KD, AD, HH, KH, NH, NK, NQ, RH, RN, SS, NN, SN, KN, NI, KI, RI, HI, SI, NG, KG, RG, RD, SD, HD, ND, KD, YG, YK, NV, HN, H*, HA, KA, N*, NA, NC, NS, RA, CI, and S*, where (*) means X¹³ is absent.

In certain embodiments, the NBD may include a plurality of RUs ordered from N-terminus to C-terminus of the NBD to recognize a target nucleic acid. For example, the NBD may include 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 RUs, where at least three of the RUs is a RU as disclosed herein. In certain aspects, the NBD may include a plurality of RUs as disclosed herein. In certain aspects, the number of RUs as disclosed herein that may be included in a NBD may be determined by the net positive charge desired for the NBD and the net charge of each RU present in the NBD. In certain aspects, the desired net positive charge of the NBD may be at least +9, at least +10, at least +11, at least +12, at least +13, at least +14, at least +15, at least +20, at least +25, at least +30, at least +35, at least +40, at least +45, at least +50, at least +55, at least +60, or more. The number of the RUs as disclosed herein that may be included in the NBD may be at least 1, 2, 3, 4, 5, 6, 7, 8, 9, 10, 11, 12, 13, 14, or more, e.g. 10-20. In certain aspects, the NBD may include one or more of the RUs disclosed herein and one or more RUs of naturally occurring transcription activator like effector (TALE) proteins, such as RUs from Xanthomonas or Ralstonia TALE proteins. RUs from TALE proteins are disclosed in, e.g., WO2019204643.

In certain aspects, the target nucleic acid may be DNA, i.e., the NBD may be a DNA-binding domain (DBD). In certain aspects, the amino acids present at positions 12 and 13 of the RUs may be selected based on the sequence of the target nucleic acid as is known for RUs from Xanthomonas or Ralstonia TALE proteins.

In certain aspects, the NBD may be associated with a functional domain. Such functional domains are further described herein. The NBD may be associated with a functional domain via a covalent interaction or via a non-covalent interaction. For example, a covalent interaction may involve conjugation of the NBD to a functional domain, e.g., a fusion protein comprising the NBD and the functional domain. A non-covalent interaction between a NBD as disclosed herein and a functional domain may involve use of binding members of a heterodimer as further explained in the next section. Briefly, the NBD may be conjugated to a first member of the heterodimer and the functional domain may be conjugated to second member of the heterodimer and the NBD and functional domain may interact via non-covalent interaction between the first and second members of the heterodimer. In certain aspects, the first member and or the second member may have a sequence that has a net positive charge (e.g., a net positive charge of at least +5, +10, +15, +20, +25, +30, or more which may then reduce the number of positively charged RUs required to impart a net positive charge on the NBD sufficient for making the NBD cell permeable.

In certain aspects, the at least three RUs present in the NBD do not comprise the amino acid sequence:

(SEQ ID NO: 27)
LTPEQVVAIACNKGGKQALKTVQRLLPVLCKPPYC;
(SEQ ID NO: 28)
LTPNQVVAIASNKGGKQALETVQRLLPVLCKPPHR;
(SEQ ID NO: 29)
LTPKQVVAIAGYKGANQALGTVQRLLPVLCKPPYG;
(SEQ ID NO: 30)
LTPKQVVAIANYKGAKQALETVQRLLPLLCKPPYG;
(SEQ ID NO: 31)
LTPKQVVAIASYKGANQALGTVQRLLPVLCKPPYG;
(SEQ ID NO: 32)
MTPKQVVAIASYKGANQALGTVQRLLPVLCKPPYG;
(SEQ ID NO: 33)
LTNDRLVALACIGGRSALNAVKDGLPNALTLIRR;
(SEQ ID NO: 34)
LTPAQVVAIASHNGGKQALKTVQRLLPVLCQAHGL;
(SEQ ID NO: 35)
LVTGQLLKIAKRGGVNAVEAVHASRNALTGAPLH;
(SEQ ID NO: 36)
LTPDQVVAIASNGGGKQALETVRRLLPVLCKPPYR;
(SEQ ID NO: 37)
LTPDQVVAIASNGGGKQALKTVQRLLPVLCKPPYS;
(SEQ ID NO: 38)
LTPNQVVAIASNHGGKQALETVQRLLPVLRKPPYG;
(SEQ ID NO: 39)
LTPEQVVAIASNKGGKQALETVQRLLPVLRKPPYG;
(SEQ ID NO: 40)
LLPHQVVAIVSNSGGKQALETVRRLLPVLCKPPYS;
(SEQ ID NO: 41)
LTPKQVVAIASYGGKQALETVQRLLPVLCKPPYG;
(SEQ ID NO: 42)
LTPKQVVAIASYGGKQSLETVQRLLPVLCKPPYG;
(SEQ ID NO: 43)
LTPKQVVAIASYKGANQALETVQRLLPVLCKPPYG;
(SEQ ID NO: 44)
LTNDRLVALACIGGRSALNAVKDGLPNALTLITR;
(SEQ ID NO: 45)
LTPNQVVAIASGIGGRQALETVHRLLPVLCKPPYG;
(SEQ ID NO: 46)
LTPNQVVAIASHDGGKQALETVQRLLPVLRKPPYG;
(SEQ ID NO: 47)
LTPEQVVAIASHGGAKQALKTVQRLLPVLCQNHGL;
(SEQ ID NO: 48)
LTPEQVVAIASHNGGKQALETVQRLLPVLCKPPYR;
(SEQ ID NO: 49)
LTPKQVVAIASHNGGKQALETVQRLLPVLCHPPYG;
(SEQ ID NO: 50)
LTPKQVVAIASHNGGKQALETVQRLLPVLCQPPYG;
(SEQ ID NO: 51)
LTPNQVVAIASHNGGKQALETVQRLLPVLCKPPYG;
(SEQ ID NO: 52)
LTRNQVVAIASHNGGKQALETVQRLLPVLCKEYGL;
(SEQ ID NO: 53)
LTPEQVVAIASKGGGKQALETVQRLLPVLCKPAYG;
(SEQ ID NO: 54)
LTPNQVVAIASKGGGKQALETVQRLLPVLCQPPYG;
(SEQ ID NO: 55)
LTPDQVVAIASKIGGKQALETVQRLLPVLCKPPYG;
(SEQ ID NO: 56)
LTPAQVVAIASNGGGKQALETVRRLLPVLCQAHGL;
(SEQ ID NO: 57)
LTPARVVAIASNGGGKQALQTVQRLLPVLCEQHGL;
(SEQ ID NO: 58)
LTPDQVVAIASNGGAKQALKTVQRLLPVLCQPPYG;
(SEQ ID NO: 59)
LTPNQVIAIASNGGGKQALETVQRLLPVLCKPPYG;
(SEQ ID NO: 60)
LTPNQVVAIASNHGGKQALETVQRLLPVLCKPPYN;
(SEQ ID NO: 61)
LTPAKVVAIASNIGGKQALETVQRLLPVLCQAHGL;
(SEQ ID NO: 62)
LTPAQVVAIACNIGGKQALETVRRLLPVLCQAHGL;
(SEQ ID NO: 63)
LTPAQVVAIASNIGGKQALETVQRLLPVLCRAHGL;
(SEQ ID NO: 64)
LTPAQVVAIASNIGGKQALETVRRLLPVLCQAHGL;
(SEQ ID NO: 65)
LTPDQVVAIARNIGGKQALETVRRLLPVLCQAHGL;
(SEQ ID NO: 66)
LTPDQVVAIASNIGGKQALKTVQRLLPVLCQAHGL;
(SEQ ID NO: 67)
LTPEQVVTIANNIGGKQALETVQRLLPVLRKPPYG;
(SEQ ID NO: 68)
LTPNQVVTIANNIGGKQALETVQRLLPVLCKPPYG;
(SEQ ID NO: 69)
LTPEQVVAIASNKGGKQALETVQRLLPVLCKPPYG;
(SEQ ID NO: 70)
LTPAQVVAIASNNGGKQALERVQRLLPVLCQAHGL;
(SEQ ID NO: 71)
LTPAQVVAIASNNGGKQALETVRRLLPVLCQAHGL;
(SEQ ID NO: 72)
LTPNQVVAIASNNGAKQALETVQRLLPVLCKPPHP;
(SEQ ID NO: 73)
LTPNQVVAIASNNGGKQALETVQRLLPVLCKPAYG;
(SEQ ID NO: 74)
LTPNQVVAIASNNGGKQALETVQRLLPVLCKPPHP;
(SEQ ID NO: 75)
LTREQVVAIASNNGGKQALETVQRLLPVLRQAHGL;
(SEQ ID NO: 76)
LTRNQVVAIVNNNGGKQALETVHRLLPVLCQPPHG;
(SEQ ID NO: 77)
LTRNQVVAIVNNNGGKQALETVHRLLPVLCQPPYG;
(SEQ ID NO: 78)
LTPAQVVAIASNSGGKQALETVQRLLPVLRQAHGL;
(SEQ ID NO: 79)
LSPNQVVAIASHNGGKPALETVQRLLPVLCKPPY;
(SEQ ID NO: 80)
LLPDQVVAIVSNNGGKLALGTVQRLLPVLCKPPY;
(SEQ ID NO: 81)
LTPAQVVAIASNGGKQALETVRRLLPVLCQAHGL;
(SEQ ID NO: 82)
LTPAQVVAIASNSGGKPALETVRRLLPVLCQAHG;
(SEQ ID NO: 83)
LTPDQVIAIVSNGGGKPALETVRRLLPVLCKHPY;
(SEQ ID NO: 84)
LTPDQVIAIVSNGGGKPALETVRRLLPVLCKPPY;
(SEQ ID NO: 85)
LTPDQVVTIASNNGGKPALETVRRLLPVLCKPPY;
(SEQ ID NO: 86)
LTPNQVVAIASNNGGKPALETVQRLLPVLCKPPY;
(SEQ ID NO: 87)
LTPVQVVAIASNGGKQALATVQRLLPVLCQAHGL;
(SEQ ID NO: 88)
LTPKQVVAIASYGGKQALETVQRLLPVLCQPPYG;
(SEQ ID NO: 89)
LSTTRVVSIACIGGRQALKAIKTHMPALRQAPYS;
(SEQ ID NO: 90)
LSTTRVVSIACIGGRQALEAIKTHMPALRQAPYS;
(SEQ ID NO: 91)
LTPQQVVAIASNTGGKQALEAVTVQLRVLRGARYG;
(SEQ ID NO: 92)
LTPQQVVAIASNTGGKRALEAVCVQLPVLRAAPYR;
(SEQ ID NO: 93)
LSTAQVVAVAGRNGGKQALEAVRAQLPALRAAPYG;
(SEQ ID NO: 94)
LSIAQVVAVASRSGGKQALEAVRAQLLALRAAPYG;
(SEQ ID NO: 95)
LSTAQVVAVASGSGGKPALEAVRAQLLALRAAPY;
(SEQ ID NO: 96)
LSTAQVVAVASGSGGKQALEAVRVQLLALRAAPYG;
(SEQ ID NO: 97)
LSTAQVVAVASGSGGKPALEAVRAQLLALRAAPYG;
(SEQ ID NO: 98)
LSTAQVVAVASGSGGKPALEAVRAQLLALRAAPYG;
(SEQ ID NO: 99)
LNTAQVVAIASHDGGKPALEAVRAKLPVLRGVPYA;
(SEQ ID NO: 100)
LSTAQVVAVASHDGGKPALEAVRKQLPVLRGVPHQ;
(SEQ ID NO: 101)
LSTAQVVAVASHDGGKPALEAVRKQLPVLRGVPHQ;
(SEQ ID NO: 102)
LSTEQVVAIASHNGGKQALEAVKAQLPVLRRAPYG;
(SEQ ID NO: 103)
LSVAQVVTIASHNGGKQALEAVRAQLLALRAAPYG;
(SEQ ID NO: 104)
LNTAQVVAIASHYGGKPALEAVWAKLPVLRGVPYA;
(SEQ ID NO: 105)
LSTAQVVAIASNGGGKQALEGIGEQLRKLRTAPYG;
(SEQ ID NO: 106)
LSPEQVVAIASNHGGKQALEAVRALFRGLRAAPYG;
(SEQ ID NO: 107)
LSTEQVVAIASNHGGKQALEAVRALFRGLRAAPYG;
(SEQ ID NO: 108)
LSTEQVVAIASNKGGKQALEAVKAQLLALRAAPYA;
(SEQ ID NO: 109)
LSTEQVVAIASNNGGKQALEAVKAQLPVLRRAPCG;
(SEQ ID NO: 110)
LSTEQVVAIASNNGGKQALEAVKAQLPVLRRAPYG;
(SEQ ID NO: 111)
LSTEQVVAVASNNGGKQALKAVKAQLLALRAAPYE;
(SEQ ID NO: 112)
LSTAQLVAIASNPGGKQALEAIRALFRELRAAPYA;
(SEQ ID NO: 113)
LSTAQLVAIASNPGGKQALEAVRALFRELRAAPYA;
(SEQ ID NO: 114)
LSTAQLVAIASNPGGKQALEAVRAPFREVRAAPYA;
(SEQ ID NO: 115)
LSTAQLVSIASNPGGKQALEAVRALFRELRAAPYA;
(SEQ ID NO: 116)
LSTAQVVAIASNPGGKQALEAVRALFRELRAAPYA;
(SEQ ID NO: 117)
LTPQQVVAIASNTGGKRALEAVRVQLPVLRAAPYE;
(SEQ ID NO: 118)
LSTAQVVAIATRSGGKQALEAVRAQLLDLRAAPYG;
(SEQ ID NO: 119)
LSTAQVVAIASSHGGKQALEAVRALFRELRAAPYG;
(SEQ ID NO: 120)
LSTAQVATIASSIGGRQALEALKVQLPVLRAAPYG;
(SEQ ID NO: 121)
LSTAQVATIASSIGGRQALEAVKVQLPVLRAAPYG;
(SEQ ID NO: 122)
FRQADIVKIASNGGSAQALNAVIKLGPTLRQRG;
(SEQ ID NO: 123)
FRQADIVKMASNGGSAQALNAVIKLGPTLRQRG;
(SEQ ID NO: 124)
FRQTDIVKMAGSGGSAQALNAVIKHGPTLRQRG;
(SEQ ID NO: 125)
FNRADIVRIAGNGGGAQALYSVRDAGPTLGKRG;
(SEQ ID NO: 126)
FSRADIVRIAGNGGGAQALYSVLDVGPTLGKRG;
(SEQ ID NO: 127)
LQRADIVKIAGNGGGAQALQAVITHRAALTQAG;
(SEQ ID NO: 128)
FSATDIVKIASNIGGAQALQAVISRRAALIQAG;
(SEQ ID NO: 129)
FSAADIVKIASNNGGAQALQAVISRRAALIQAG;
(SEQ ID NO: 130)
FTLTDIVKMAGNNGGAQALKVVLEHGPTLRQRG.
(SEQ ID NO: 131)
FNTEQIVRMVSHDGGSLNLKAVKKYHDALRERK;
(SEQ ID NO: 132)
LDRQQILRIASHDGGSKNIAAVQKFLPKLMNFG;
(SEQ ID NO: 133)
FSAKHIVRIAAHIGGSLNIKAVQQAQQALKELG;
(SEQ ID NO: 134)
LGHKELIKIAARNGGGNNLIAVLSCYAKLKEMG;
(SEQ ID NO: 135)
FNAEQIVRMVSHKGGSKNLALVKEYFPVFSSFH;
(SEQ ID NO: 136)
FNAEQIVRMVSHKGGSKNLALVKEYFPVFSSFH;
(SEQ ID NO: 137)
FNAEQIVSMVSNGGGSLNLKAVKKYHDALKDRG;
(SEQ ID NO: 138)
LEPKDIVSIASHIGATQAITTLLNKWAALRAKG;
or
(SEQ ID NO: 139)
FNRASIVKIAGNSGGAQALQAVLKHGPTLDERG.

In other aspects, the NBD in addition to including at least three (e.g., 10-20) non-naturally occurring RU having a net positive charge of at least +1, where the RU is derived from the sequence of SEQ ID NO:1 and include at least one amino acid substitution as provided in the foregoing section, the NBD may include RUs derived from naturally occurring proteins comprising such RUs and selected because these RUs comprise an amino acid sequence that has a net charge of at least +1. Such RUs may have an amino acid sequence as set forth in any one of SEQ ID NO: 27-139.

In certain aspects, one or more RUs in a NBD may be at least 80%, 85%, 90%, 91%, 92%, 93%, 94%, 95%, 96%, 97%, 98%, or 99%, or a 100% identical to a RU provided herein. Percent identity between a pair of sequences may be calculated by multiplying the number of matches in the pair by 100 and dividing by the length of the aligned region, including gaps. Identity scoring only counts perfect matches and does not consider the degree of similarity of amino acids to one another. Only internal gaps are included in the length, not gaps at the sequence ends.

Percent Identity=(Matches×100)/Length of aligned region (with gaps)

Also disclosed herein are polypeptides that are at least 80%, 85%, 90%, 91%, 92%, 93%, 94%, 95%, 96%, 97%, 98%, or 99%, or a 100% identical to an amino acid sequence disclosed herein.

The phrase “conservative amino acid substitution” refers to substitution of amino acid residues within the following groups: 1) L, I, M, V, F; 2)R, K; 3) F, Y, H, W, R; 4) G, A, T, S; 5) Q, N; and 6) D, E. Conservative amino acid substitutions may preserve the activity of the protein by replacing an amino acid(s) in the protein with an amino acid with a side chain of similar acidity, basicity, charge, polarity, or size of the side chain.

Guidance for substitutions, insertions, or deletions may be based on alignments of amino acid sequences of proteins from different species or from a consensus sequence based on a plurality of proteins having the same or similar function.

In certain aspects, the disclosed NBD may include a nuclear localization sequence (NLS) to facilitate entry into an organelle of a cell, e.g. the nucleus of a cell, e.g., an animal or a plant cell. In certain aspects, the disclosed NBD may include a half-RU or a partial RU that is 15-20 amino acid long sequence. Such a half-RU may be included after the last RU present in the NBD and may be derived from a RU identified in Xanthomonas or Ralstonia TALE protein. This half-RU may not be modified to provide a net positive charge to the RU. The half-RU may comprise a nucleic acid sequence at least 80% or more (at least 90%, at least 95%, at least 96%, at least 97%, at least 98%, or at least 99%) identical to the amino acid sequence: LTPEQVVAIASX¹²X¹³GGRPALE (SEQ ID NO:186). In certain aspects, the disclosed NBD may include an N-terminal domain. The N-terminal domain may be the N-cap domain or a fragment thereof from TALE proteins like those expressed in Burkholderia, Paraburkholderia, or Xanthomonas. In certain aspects, the disclosed NBD may include a C-terminal domain. The C-terminal domain may be a C-cap domain or a fragment thereof from TALE proteins like those expressed in Burkholderia, Paraburkholderia, or Xanthomonas.

### Positively Charged Heterodimer Pairs

The present disclosure provides heterodimerization domains that are binding members of a heterodimer pair and have been modified by amino acid substitution to introduce positively charged amino acids thereby increasing the positive charge of the binding members.

In certain aspects, the binding members of a heterodimer pair are referred to as 37A and 37B. The sequences of the unmodified proteins 37A and 37B are as follows:

37A_Unmodified:
(SEQ ID NO: 2)
DSDEHLKKLKTFLENLRRHLDRLDKHIKQLRDILSENPEDERVKDVID
LSERSVRIVKTVIKIFEDSVRKKE
37B_Unmodified:
(SEQ ID NO: 3)
MDDKELDKLLDTLEKILQTATKIIDDANKLLEKLRRSERKDPKVVETY
VELLKRHEKAVKELLEIAKTHAKKVE

The underlined residues indicate amino acids that can be substituted with an amino acid with a positively charged side chain, e.g., K, R, or H, without significantly reducing dimerization of 37A and 37B.

In certain aspects, 1-14, e.g., 3-14, 5-14, 8-14, 5-12, 5-9, such as, 3, 5, 8, 9, 12, or 14 amino acids of the 37A protein may be substituted with an amino acid with a positively charged side chain. For example, a positively charged first member of a heterodimer pair may have an amino acid sequence that is about 72 amino acids long and is at least 75% identical to the sequence of the unmodified 37A protein (SEQ ID NO:2) and comprises at least one of the following amino acid substitutions relative to the sequence of the unmodified 37A protein: D3K/R/H; E4K/R/H; T11K/R/H; D24K/R/H; D32K/R/H; S35K/R/H; E39K/R/H; D40K/R/H; E41K/R/H; D45K/R/H; D48K/R/H; L49K/R/H; T59K/R/H; and D66K/R/H.

In certain aspects, a positively charged first member of a heterodimer pair may have an amino acid sequence that is at least 75% identical (e.g., at least 80%) to the sequence of the unmodified 37A protein (SEQ ID NO:2) and comprises at least one, at least two, at least three, at least four, at least five, at least six, at least seven, at least eight, at least nine, at least ten, at least eleven, at least twelve, at least thirteen, or all of the following amino acid substitutions relative to the sequence of the unmodified 37A protein: D3K; E4K; T11K; D24K; D32K; S35K; E39K; D40K; E41K; D45K; D48K; L49K; T59K; and D66K. In certain aspects, a positively charged first member of a heterodimer pair may have the amino acid sequence of SEQ ID NO:2 but with at least one, at least two, at least three, at least four, at least five, at least six, at least seven, at least eight, at least nine, at least ten, at least eleven, at least twelve, at least thirteen, or all of the following amino acid substitutions relative to the sequence of SEQ ID NO:2: D3K; E4K; T11K; D24K; D32K; S35K; E39K; D40K; E41K; D45K; D48K; L49K; T59K; and D66K.

In certain aspects, a positively charged 37A protein may have an amino acid sequence as follows:

(SEQ ID NO: 4)
DSDEHLKKLKKFLENLRRHLDRLKKHIKQLRDILSENPEDKRVKDVID
LSERSVRIVKTVIKIFEDSVRKKE;
(SEQ ID NO: 5)
DSKEHLKKLKKFLENLRRHLDRLKKHIKQLRKILSENPEDKRVKDVID
LSERSVRIVKTVIKIFEDSVRKKE;
(SEQ ID NO: 6)
DSKKHLKKLKKFLENLRRHLDRLKKHIKQLRKILKENPEDKRVKDVID
LSERSVRIVKKVIKIFEDSVRKKE;
(SEQ ID NO: 7)
DSKKHLKKLKKFLENLRRHLDRLKKHIKQLRKILKENPEDKRVKDVID
KSERSVRIVKKVIKIFEDSVRKKE;
(SEQ ID NO: 8)
DSKKHLKKLKKFLENLRRHLDRLKKHIKQLRKILKENPKDKRVKDVID
KSERSVRIVKKVIKIFEKSVRKKE;
or
(SEQ ID NO: 9)
DSKKHLKKLKKFLENLRRHLDRLKKHIKQLRKILKENPKKKRVKKVIK
KSERSVRIVKKVIKIFEKSVRKKE;

Amino acid substitutions relative to the unmodified 37A protein are indicated by underlining.

In certain aspects, 1-13, e.g., 3-9, 5-9, or 8-9, such as, 3, 5, 7, 8, or 9 amino acids of the 37B protein may be substituted with an amino acid with a positively charged side chain e.g., K, R, or H. For example, a positively charged first member of a heterodimer pair may have an amino acid sequence that is about 74 amino acids long and is at least 75% identical (e.g., at least 80% or 85% identical) to the sequence of the unmodified 37B protein (SEQ ID NO:3) and comprises at least one (e.g., at least 2, at least 3, at least 4, at least 5, at least 6, at least 7, at least 8, at least 9, at least 10, at least 11, at least 12, or all) of the following amino acid substitutions relative to the sequence of the unmodified 37B protein: D2K/R/H; D3K/R/H; E5K/R/H; T12K/R/H; T19K/R/H; D26K/R/H; E38K/R/H; D41K/R/H; E46K/R/H; E56K/R/H; E61K/R/H; T68K/R/H; and E74K/R/H.

In certain aspects, a positively charged second member of a heterodimer pair may have the amino acid sequence of SEQ ID NO:3 but with at least one (e.g., at least 2, at least 3, at least 4, at least 5, at least 6, at least 7, at least 8, at least 9, at least 10, at least 11, at least 12, or all) of the following amino acid substitutions relative to the sequence of SEQ ID NO:3: D2K/R/H; D3K/R/H; E5K/R/H; T12K/R/H; T19K/R/H; D26K/R/H; E38K/R/H; D41K/R/H; E46K/R/H; E56K/R/H; E61K/R/H; T68K/R/H; and E74K/R/H.

In certain aspects, a positively charged 37B protein may have an amino acid sequence as follows:

(SEQ ID NO: 10)
MKDKELDKLLDTLEKILQKATKIIDDANKLLEKLRRSERKKPKVVETY
VELLKRHEKAVKELLEIAKTHAKKVE;
(SEQ ID NO: 16)
MDDKKLDKLLDKLEKILQTATKIIDDANKLLEKLRRSERKDPKVVKTY
VELLKRHEKAVKELLEIAKTHAKKVE;
(SEQ ID NO: 12)
MKDDKELDKLLDTLEKILQTATKIIDKANKLLEKLRRSKRKDPKVVET
YVELLKRHEKAVKELLEIAKKHAKKVE;
(SEQ ID NO: 13)
MKDKELDKLLDKLEKILQKATKIIDKANKLLEKLRRSERKKPKVVKTY
VELLKRHEKAVKELLEIAKTHAKKVE;
(SEQ ID NO: 14)
MKDKKLDKLLDKLEKILQKATKIIDKANKLLEKLRRSKRKKPKVVKTY
VELLKRHEKAVKELLEIAKTHAKKVE;
or
(SEQ ID NO: 15)
MKKDKKLDKLLDKLEKILQKAKIIDKANKLLEKLRRSKRKKPKVVKTY
VELLKRHEKAVKELLEIAKTHAKKVE

Amino acid substitutions relative to the unmodified 37B protein are indicated by underlining.

In certain aspects, a positively charged first binding member or positively charged second binding member of a heterodimer may be fused to a nuclear localization sequence (NLS). The NLS may be a positively charged nuclear localization sequence, e.g., PKKKRKV (SEQ ID NO:173).

In certain aspects, a positively charged first binding member or positively charged second binding member of a heterodimer may be fused to a NBD or a functional domain. For example, a positively charged first binding member may be fused to a NBD and a positively charged second binding member of the heterodimer may be fused to a functional domain. The NBD and the functional domain may be as described herein or as are known in the art. The first or the second member may be fused to the N- or the C-terminus of the NBD or the functional domain. In certain aspects, the NBD may be a transcription activator-like effector (TALE), modular animal pathogen nucleic acid binding domain, zinc finger protein, or single-guide RNA. Modular animal pathogen nucleic acid binding domain may be derived from DNA binding RUs identified in proteins from animal pathogens, such as, Legionella quateirensis, Burkholderia, Paraburkholderia, or Francisella.

In certain aspects, instead of or in addition to substituting in amino acids with positively charged side chain in the sequence of a first binding member and/or a second binding member of a heterodimer as disclosed herein, a binding member of a heterodimer may be fused to a nucleic acid binding domain or a functional domain via a positively charged linker. In certain aspects, the positively charged linker may be include at least 4, at least 5, or at least 6 amino acids with a positively charged side chain. In certain aspects, a positively charged linker may comprise the sequence: GKGSKGKGKGK (SEQ ID NO: 140), GKGSKGKGKGKGSK (SEQ ID NO: 141), or GKGSKGKGKGKMDAKSLTAWS (SEQ ID NO: 162).

In certain aspects, a first or a second binding member of a heterodimer may be conjugated to the N- or C-terminus of a nucleic acid binding domain or a functional domain with or without a linker. The linker, if present, may have a net neutral charge or may have a net positive charge.

In certain aspects, a heterodimer comprising the first binding member and the second binding member as provided herein is disclosed. The first binding member and/or the second binding member may be fused to a NBD or a functional domain.

In certain aspects, the heterodimer may include a first binding member and a second binding member as provided herein, where the first binding member is fused to a functional domain (e.g., to the N-terminus of the functional domain) and the second binding member is fused to a DNA binding domain (e.g., to the C-terminus of the DNA binding domain).

In certain aspects, the heterodimer may include a first binding member and a second binding member as provided herein, where the second binding member is fused to a functional domain (e.g., to the N-terminus of the functional domain) and the first binding member is fused to a DNA binding domain e.g., to the C-terminus of the DNA binding domain.

In certain aspects, the first binding member as disclosed herein comprises a net charge of at least +15 (e.g., at least +20, +25, +30, or more). In certain aspects, the second binding member comprises a net charge of at least +15 (e.g., at least +20, +25, +30, or more). In certain aspects, the first binding member and the second binding member each comprise a net charge of at least +15 (e.g., at least +20, +25, +30, or more).

In certain aspects, the second binding member may have an amino acid sequence at least 80%, at least 90%, at least 95%, at least 96%, at least 97%, at least 98%, %, at least 99%, or 100% identical to the amino acid sequence of:

>37B-linker-KRAB-net5-1
(SEQ ID NO: 142)
MKDKELDKLLDTLEKILQKATKIIDDANKLLEKLRRSERKKPKVVETYVE
LLKRHEKAVKELLEIAKTHAKKVEGSGGGGGMDAKSLTAWSRTLVTFKDVFVDFTREEW
KLLDTAQQIVYRNVMLENYKNLVSLGYOLTKPDVILRLEKGEEP
>37B-linker-KRAB-net5-2
(SEQ ID NO: 143)
MDDKKLDKLLDKLEKILQTATKIIDDANKLLEKLRRSERKDPKVVKTYV
ELLKRHEKAVKELLEIAKTHAKKVEGSGGGGGMDAKSLTAWSRTLVTFKDVFVDFTREE
WKLLDTAQQIVYRNVMLENYKNLVSLGYQLTKPDVILRLEKGEEP
>37B-linker-KRAB-net5-3
(SEQ ID NO: 144)
MKDDKELDKLLDTLEKILQTATKIIDKANKLLEKLRRSKRKDPKVVETY
VELLKRHEKAVKELLEIAKKHAKKVEGSGGGGGMDAKSLTAWSRTLVTFKDVFVDFTRE
EWKLLDTAQQIVYRNVMLENYKNLVSLGYOLTKPDVILRLEKGEEP
>37B-linker-KRAB-net10
(SEQ ID NO: 145)
MKDKELDKLLDKLEKILQKATKIIDKANKLLEKLRRSERKKPKVVKTYV
ELLKRHEKAVKELLEIAKTHAKKVEGSGGGGGMDAKSLTAWSRTLVTFKDVFVDFTREE
WKLLDTAQQIVYRNVMLENYKNLVSLGYOLTKPDVILRLEKGEEP
>37B-linker-KRAB-net15
(SEQ ID NO: 146)
MKDKKLDKLLDKLEKILQKATKIIDKANKLLEKLRRSKRKKPKVVKTYV
ELLKRHEKAVKELLEIAKTHAKKVEGSGGGGGMDAKSLTAWSRTLVTFKDVFVDFTREE
WKLLDTAQQIVYRNVMLENYKNLVSLGYOLTKPDVILRLEKGEEP
>37B-linker-KRAB-net20
(SEQ ID NO: 147)
MKKDKKLDKLLDKLEKILQKATKIIDKANKLLEKLRRSKRKKPKVVKTY
VELLKRHEKAVKELLEIAKTHAKKVEGKGSKGKGKGKMDAKSLTAWSRTLVTFKDVFV
DFTREEWKLLDTAQQIVYRNVMLENYKNLVSLGYQLTKPDVILRLEKGEEP

The amino acid substitutions relative to the unmodified 37B protein are underlined; the linker sequence is in bold font; and KRAB sequence is italicized. In certain aspects, the 37B-linker-KRAB polypeptide is fused to a NLS.

In certain aspects, instead of using the 37A and 37B proteins (or modified variants thereof) to mediate interaction between a nucleic acid binding domain and a functional domain, the binding members A1::B1; A2::B2; A3::B3; A4::B4, and A5::B5 of a heterodimer may be used. Sequences for these heterodimers are as follows:

A1:
(SEQ ID NO: 148)
PTDEVIEVLKELLRIHRENLRVNEEIVEVNERASRVTDREELERLLRRS
NELIKRSRELNEESKKLIEKLERLAT;
and
B1:
(SEQ ID NO: 149)
DNEEIIKEARRVVEEYKKAVDRLEELVRRAENAKHASEKELKDIVREIL
RISKELNKVSERLIELWERSQERAR;
or
A2:
(SEQ ID NO: 150)
TAEELLEVHKKSDRVTKEHLRVSEEILKVVEVLTRGEVSSEVLKRVLRK
LEELTDKLRRVTEEQRRVVEKLN;
and
B2:
(SEQ ID NO: 151)
DLEDLLRRLRRLVDEQRRLVEELERVSRRLEKAVRDNEDERELARLSRE
HSDIQDKHDKLAREILEVLKRLLERTE;
or
A3:
(SEQ ID NO: 152)
PEDDVVRIIKEDLESNREVLREQKEIHRILELVTRGEVSEEAIDRVLKR
DLLKKQKESTDKARKVVEERR;
QEand
B3:
(SEQ ID NO: 153)
DEVRLITEWLKLSEESTRLLKELVELTRLLRNNVPNVEEILREHERISR
ELERLSRRLKDLADKLERTRR;
or
A4
(SEQ ID NO: 154)
DEEDHLKKLKTHLEKLERHLKLLEDHAKKLEDILKERPEDSAVKESIDE
LRRSIELVRESIEIFRQSVEEEE;
and
B4:
(SEQ ID NO: 155)
GDVKELTKILDTLTKILETATKVIKDATKLLEEHRKSDKPDPRLIETHK
KLVEEHETLVRQHKELAEEHLKRTR;
or
A5:
(SEQ ID NO: 156)
MKKDKKLDKLLDKLEKILQKATKIIDKANKLLEKLRRSKRKKPKVVKTY
VELLKRHEKAVKELLEIAKTHAKKVE;
and
B5:
(SEQ ID NO: 157)
MKKDKKLDKLLDKLEKILQKATKIIDKANKLLEKLRRSKRKKPKVVKTY
VELLKRHEKAVKELLEIAKTHAKKVE

In certain aspects, one or both binding members may include amino acid substitutions replacing an amino acid with a neutral or a negatively charged side chain with K, R, or H. In certain aspects, a first binding member may be conjugated to a nucleic acid binding domain and a second binding member of the same binding pair may be conjugated to a functional domain via a positively charged linker.

Polypeptides disclosed herein include a polypeptide comprising at least 80% identity, at least 85% identity, at least 90% identity, at least 95% identity, at least 96% identity, at least 97% identity, at least 98% identity, at least 99% identity, or a 100% identity to any one of the polypeptide sequences disclosed herein, including the polypeptides or fragments thereof disclosed in the examples section.

### Functional Domains

A NBD as disclosed herein can be associated with a functional domain as described in the preceding sections. The functional domain can provide different types of activity, such as genome editing, gene regulation (e.g., activation or repression), or visualization of a genomic locus via imaging. In certain aspects, the functional domain is heterologous to the NBD. Heterologous in the context of a functional domain and a NBD as used herein indicates that these domains are derived from different sources and do not exist together in nature.

A. Genome Editing Domains

A NBD as disclosed herein can be associated with a nuclease, wherein the NBD provides specificity and targeting and the nuclease provides genome editing functionality. In some embodiments, the nuclease can be a cleavage half domain, which dimerizes to form an active full domain capable of cleaving DNA. In other embodiments, the nuclease can be a cleavage domain, which is capable of cleaving DNA without needing to dimerize. For example, a nuclease comprising a cleavage half domain can be an endonuclease, such as FokI or BfiI. In some embodiments, two cleavage half domains (e.g., FokI or BfiI) can be fused together to form a fully functional single cleavage domain. When half cleavage domains are used as the nuclease, two MAP-NBDs can be engineered, the first MAP-NBD binding to a top strand of a target nucleic acid sequence and comprising a first FokI cleavage half domain and a second MAP-NBD binding to a bottom strand of a target nucleic acid sequence and comprising a second FokI half cleavage domain. In some embodiments, the nuclease can be a type IIS restriction enzyme, such as FokI or BfiI.

In some embodiments, a cleavage domain capable of cleaving DNA without need to dimerize may be a meganuclease. Meganucleases are also referred to as homing endonucleases. In some embodiments, the meganuclease may be I-Anil or I-OnuI.

A nuclease domain fused to a NBD can be an endonuclease or an exonuclease. An endonuclease can include restriction endonucleases and homing endonucleases. An endonuclease can also include S1 Nuclease, mung bean nuclease, pancreatic DNase I, micrococcal nuclease, or yeast HO endonuclease. An exonuclease can include a 3′-5′ exonuclease or a 5′-3′ exonuclease. An exonuclease can also include a DNA exonuclease or an RNA exonuclease. Examples of exonuclease includes exonucleases I, II, III, IV, V, and VIII; DNA polymerase I, RNA exonuclease 2, and the like.

A nuclease domain fused to a NBD as disclosed herein can be a restriction endonuclease (or restriction enzyme). In some instances, a restriction enzyme cleaves DNA at a site removed from the recognition site and has a separate binding and cleavage domains. In some instances, such a restriction enzyme is a Type IIS restriction enzyme.

A nuclease domain fused to a NBD as disclosed herein can be a Type IIS nuclease. A Type IIS nuclease can be FokI or BfiI. In some cases, a nuclease domain fused to a MAP-NBD (e.g., L. quateirensis, Burkholderia, Paraburkholderia, or Francisella-derived) is FokI. In other cases, a nuclease domain fused to a MAP-NBD (e.g., L. quateirensis, Burkholderia, Paraburkholderia, or Francisella-derived) is BfiI.

FokI can be a wild-type FokI or can comprise one or more mutations. In some cases, FokI can comprise 1, 2, 3, 4, 5, 6, 7, 8, 9, 10, or more mutations. A mutation can enhance cleavage efficiency. A mutation can abolish cleavage activity. In some cases, a mutation can modulate homodimerization. For example, FokI can have a mutation at one or more amino acid residue positions 446, 447, 479, 483, 484, 486, 487, 490, 491, 496, 498, 499, 500, 531, 534, 537, and 538 to modulate homodimerization.

In some instances, a FokI cleavage domain is, for example, as described in Kim et al. “Hybrid restriction enzymes: Zinc finger fusions to FokI cleavage domain,” PNAS 93: 1156-1160 (1996). In some cases, a FokI cleavage domain described herein is a FokI of SEQ ID NO: 11 (TABLE 2). In other instances, a FokI cleavage domain described herein is a FokI, for example, as described in U.S. Pat. No. 8,586,526.

TABLE 2
illustrates an exemplary FokI sequence that can be used herein with a
method or system described herein.

| SEQ ID NO | FokI Sequence |
|---|---|
| SEQ ID NO: 11 | QLVKSELEEKKSELRHKLKYVPHEYIELIEIARNSTQDRILEMKVMEFF |
| | MKVYGYRGKHLGGSRKPDGAIYTVGSPIDYGVIVDTKAYSGGYNLPIG |
| | QADEMQRYVEENQTRNKHINPNEWWKVYPSSVTEFKFLFVSGHFKGN |
| | YKAQLTRLNHITNCNGAVLSVEELLIGGEMIKAGTLTLEEVRRKENNG |
| | EINF |

A NBD can be linked to a functional group that modifies DNA nucleotides, or example an adenosine deaminase.

B. Regulatory Domains

As another example, NBD as disclosed herein can be linked to a gene regulating domain. A gene regulation domain can be an activator or a repressor. For example, a NBD as disclosed herein can be linked to an activation domain, such as VP16, VP64, p65, p300 catalytic domain, TET1 catalytic domain, TDG, Ldb1 self-associated domain, SAM activator (VP64, p65, HSF1), or VPR (VP64, p65, Rta). The terms “activator,” “activation domain” and “transcriptional activator” are used interchangeably to refer to a polypeptide that increases expression of a gene. Alternatively, a NBD can be linked to a repressor, such as KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2. The terms “repressor,” “repressor domain,” and “transcriptional repressor” are used herein interchangeably to refer to a polypeptide that decreases expression of a gene.

In some embodiments, a NBD as disclosed herein can be linked to a DNA modifying protein, such as DNMT3a. A NBD can be linked to a chromatin-modifying protein, such as lysine-specific histone demethylase 1 (LSD1). A NBD can be linked to a protein that is capable of recruiting other proteins, such as KRAB. The DNA modifying protein (e.g., DNMT3a) and proteins capable of recruiting other proteins (e.g., KRAB) can serve as repressors of transcription. Thus, NBD linked to a DNA modifying protein (e.g., DNMT3a) or a domain capable of recruiting other proteins (e.g., KRAB, a domain found in transcriptional repressors, such as Kox1) can provide gene repression functionality, can serve as transcription factors, wherein the NBD provides specificity and targeting and the DNA modifying protein and the protein capable of recruiting other proteins provides gene repression functionality, which can be referred to as an engineered genomic regulatory complex or a NBD-gene regulator (NBD-GR) and, more specifically, as a NBD-transcription factor (NBD-TF).

In some embodiments, expression of the target gene can be reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 92%, at least 95%, at least 97%, or at least 99% by using a DNA binding domain fused to a repression domain (e.g., a MAP-NBD-TF) of the present disclosure as compared to non-treated cells. In some embodiments, expression of a checkpoint gene can be reduced by over 90% by using a MAP-NBD-TF of the present disclosure as compared to non-treated cells.

In some embodiments, repression of the target gene with a DNA binding domain fused to a repression domain (e.g., a NBD-TF) of the present disclosure and subsequent reduced expression of the target gene can last for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, or at least 28 days. In some embodiments, repression of the target gene with a MAP-NBD-TF of the present disclosure and subsequent reduced expression of the target gene can last for 1 days to 3 days, 3 days to 5 days, 5 days to 7 days, 7 days to 9 days, 9 days to 11 days, 11 days to 13 days, 13 days to 15 days, 15 days to 17 days, 17 days to 19 days, 19 days to 21 days, 21 days to 23 days, 23 days to 25 days, or 25 days to 28 days.

In various aspects, the present disclosure provides a method of identifying a target binding site in a target gene of a cell, the method comprising: (a) contacting a cell with an engineered transcriptional repressor comprising a DNA binding domain, a repressor domain, and a linker; (b) measuring expression of the target gene; and (c) determining expression of the target gene is repressed by at least 50%, at least 60%, at least 70%, at least 80%, at least 85%, at least 90%, at least 92%, at least 95%, at least 97%, or at least 99% for at least 3 days, wherein the target gene is selected from: a checkpoint gene and a T cell surface receptor.

In some aspects, expression of the target gene is repressed in at least 75%, at least 80%, at least 85%, at least 90%, at least 95%, or at least 99% of a plurality of the cells. In some aspects, the engineered genomic regulatory complex is undetectable after at least 3 days. In some aspects, determining the engineered genomic regulatory complex is undetectable is measured by qPCR, imaging of a FLAG-tag, or a combination thereof. In some aspects, the measuring expression of the target gene comprises flow cytometry quantification of expression of the target gene.

In some embodiments, repression of the target gene with a DNA binding domain targeting a repression domain (e.g., a NBD fused to TF or NBD-1st heterodimerization domain:: 2^{nd} heterodimerization domain:functional domain) of the present disclosure can last even after the DNA binding domain-TF becomes undetectable. The genome modifying proteins can become undetectable after at least 3 days. In some embodiments, the genome modifying proteins can become undetectable after at least 1 day, at least 2 days, at least 3 days, at least 4 days, at least 5 days, at least 6 days, at least 1 week, at least 2 weeks, at least 3 weeks, or at least 4 weeks. In some embodiments, qPCR or imaging via a tag can be used to confirm that the genome modifying proteins are no longer detectable.

C. Imaging Moieties

In certain aspects, the functional domain may be an imaging domain, e.g, a fluorescent protein, biotinylation reagent, tag (e.g., 6×-His or HA). A NBD can be linked to a fluorophore, such as Hydroxycoumarin, methoxycoumarin, Alexa fluor, aminocoumarin, Cy2, FAM, Alexa fluor 488, Fluorescein FITC, Alexa fluor 430, Alexa fluor 532, HEX, Cy3, TRITC, Alexa fluor 546, Alexa fluor 555, R-phycoerythrin (PE), Rhodamine Red-X, Tamara, Cy3.5, Rox, Alexa fluor 568, Red 613, Texas Red, Alexa fluor 594, Alexa fluor 633, Allophycocyanin, Alexa fluor 633, Cy5, Alexa fluor 660, Cy5.5, TruRed, Alexa fluor 680, Cy7, GFP, or mCHERRY.

### Positively Charged Fusion Proteins

As described in the preceding sections, the polypeptide comprising the at least three RUs described above may be conjugated to the first binding member or the second binding member.

The polypeptide may include a NLS as described herein. In certain embodiments, the polypeptide may include one or more purification/detection tags, such as, His-tag, GST-tag, HA tag, SPOT-tag®, T7 tag, and/or V5 tag.

The first binding member may include a NLS as described herein. In certain embodiments, the first binding member may include one or more purification/detection tags, such as, His-tag, GST-tag, HA tag, SPOT-tag®, T7 tag, and/or V5 tag.

The second binding member may include a NLS as described herein. In certain embodiments, the second binding member may include one or more purification/detection tags, such as, His-tag, GST-tag, HA tag, SPOT-tag®, T7 tag, and/or V5 tag.

In certain embodiments, a polypeptide of the disclosure may comprise of [N-terminal tag]--[DNA binding domain]--[positively charged or uncharged linker]--[Heterodimer A/B], arranged from N-terminus to C-terminus.

In certain embodiments, a polypeptide of the disclosure may comprise of:

[N-terminal tag]--[DNA binding domain]--[positively charged or uncharged linker]--[Heterodimer A]--[positively charged or uncharged linker]--[Heterodimer A]--[positively charged or uncharged linker]--[Heterodimer A]; or

[N-terminal tag]--[DNA binding domain]--[positively charged or uncharged linker]--[Heterodimer B]--[positively charged or uncharged linker]--[Heterodimer B]--[positively charged or uncharged linker]--[Heterodimer B], arranged from N-terminus to C-terminus.

The [N-terminal tag] can include one or more of a purification/detection tag and a NLS.

In certain embodiments, the [N-terminal tag] may include one or more of a purification/detection tag, e.g., His-tag (e.g., 6×-10× His, such as 6×-His tag or 9×-His tag), SPOT-tag, T7 tag, ad V5 tag and a NLS.

The [DNA binding domain] is a NBD as described in the preceding sections. The [Heterodimer A] may be the first binding member as described in the preceding sections. The [Heterodimer B] may be the second binding member as described in the preceding sections.

The [positively charged or uncharged linker] may be as described in the preceding sections. The uncharged linker may be a sequence comprising the amino acid sequence: GGG, GGGGGMDAKSLTAWS (SEQ ID NO:163), or GGGMDAKSLTAWS (SEQ ID NO:164). A positively charged linker may be a sequence comprising the amino acid sequence: GSKGKGKGK (SEQ ID NO:165) or GSKGKGKGKMDAKSLTAWS (SEQ ID NO:166).

In certain embodiments, the first binding member of the disclosure may comprise of: [Heterodimer A]--[positively charged or uncharged linker]--[functional domain], arranged from N-terminus to C-terminus. [Heterodimer A] may be the first binding member.

In certain embodiments, the second binding member of the disclosure may comprise of: [Heterodimer B]--[positively charged or uncharged linker]--[functional domain], arranged from N-terminus to C-terminus. [Heterodimer B] may be the second binding member.

The first and second binding members may further include a [N-terminal tag] which may be a purification/detection tag that may be cleavably conjugated to the first and second binding member. Cleavage of the tag may be achieved by using a protease cleavage site.

The [positively charged or uncharged linker] may be as described in the preceding sections. The uncharged linker may be a sequence comprising the amino acid sequence: GGG, GGGGGMDAKSLTAWS (SEQ ID NO:163), or GGGMDAKSLTAWS (SEQ ID NO:164). A positively charged linker may be a sequence comprising the amino acid sequence: GSKGKGKGK (SEQ ID NO:165), GGGSKGKGKGKMDAKSLTAWS (SEQ ID NO:167), or GSKGKGKGKMDAKSLTAWS (SEQ ID NO:166).

As explained in detail herein, (i) the polypeptide fused to a first binding member and (ii) the second binding member fused to a functional domain are components that associate via the first and second binding members to locate the functional domain to the target gene to which the polypeptide binds.

### Targets

In some aspects, described herein include methods of modifying the genetic material of a target cell utilizing a NBD and a functional domain described herein. A target cell can be a eukaryotic cell or a prokaryotic cell. A target cell can be an animal cell or a plant cell. An animal cell can include a cell from a marine invertebrate, fish, insects, amphibian, reptile, or mammal. A mammalian cell can be obtained from a primate, ape, equine, bovine, porcine, canine, feline, or rodent. A mammal can be a primate, ape, dog, cat, rabbit, ferret, or the like. A rodent can be a mouse, rat, hamster, gerbil, hamster, chinchilla, or guinea pig. A bird cell can be from a canary, parakeet or parrots. A reptile cell can be from a turtle, lizard or snake. A fish cell can be from a tropical fish. For example, the fish cell can be from a zebrafish (e.g., Danio rerio). A worm cell can be from a nematode (e.g., C. elegans). An amphibian cell can be from a frog. An arthropod cell can be from a tarantula or hermit crab.

A mammalian cell can also include cells obtained from a primate (e.g., a human or a non-human primate). A mammalian cell can include an epithelial cell, connective tissue cell, hormone secreting cell, a nerve cell, a skeletal muscle cell, a blood cell, an immune system cell, or a stem cell.

Exemplary mammalian cells can include, but are not limited to, 293A cell line, 293FT cell line, 293F cells, 293 H cells, HEK 293 cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, NC-HIMT cell line, PC12 cell line, primary cells (e.g., from a human) including primary T cells, primary hematopoietic stem cells, primary human embryonic stem cells (hESCs), and primary induced pluripotent stem cells (iPSCs).

In some embodiments, a NBD of the present disclosure can be used to modify a target cell. The target cell can itself be unmodified or modified. For example, an unmodified cell can be edited with a NBD of the present disclosure to introduce an insertion, deletion, or mutation in its genome. In some embodiments, a modified cell already having a mutation can be repaired with a NBD of the present disclosure.

In some instances, a target cell is a cell comprising one or more single nucleotide polymorphism (SNP). In some instances, a NBD-nuclease described herein is designed to target and edit a target cell comprising a SNP.

In some cases, a target cell is a cell that does not contain a modification. For example, a target cell can comprise a genome without genetic defect (e.g., without genetic mutation) and a NBD-nuclease described herein can be used to introduce a modification (e.g., a mutation) within the genome.

In some cases, a target cell is a cancerous cell. Cancer can be a solid tumor or a hematologic malignancy. The solid tumor can include a sarcoma or a carcinoma. Exemplary sarcoma target cell can include, but are not limited to, cell obtained from alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, or telangiectatic osteosarcoma.

Exemplary carcinoma target cell can include, but are not limited to, cell obtained from anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

Alternatively, the cancerous cell can comprise cells obtained from a hematologic malignancy. Hematologic malignancy can comprise a leukemia, a lymphoma, a myeloma, a non-Hodgkin's lymphoma, or a Hodgkin's lymphoma. In some cases, the hematologic malignancy can be a T-cell based hematologic malignancy. Other times, the hematologic malignancy can be a B-cell based hematologic malignancy. Exemplary B-cell based hematologic malignancy can include, but are not limited to, chronic lymphocytic leukemia (CLL), small lymphocytic lymphoma (SLL), high-risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. Exemplary T-cell based hematologic malignancy can include, but are not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some cases, a cell can be a tumor cell line. Exemplary tumor cell line can include, but are not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

In some embodiments, described herein include methods of modifying a target gene utilizing a NBD described herein. In some embodiments, genome editing can be performed by fusing a nuclease of the present disclosure with a DNA binding domain for a particular genomic locus of interest. Genetic modification can involve introducing a functional gene for therapeutic purposes, knocking out a gene for therapeutic gene, or engineering a cell ex vivo (e.g., HSCs or CAR T cells) to be administered back into a subject in need thereof. For example, the genome editing complex can have a target site within PDCD1, CTLA4, LAG3, TET2, BTLA, HAVCR2, CCR5, CXCR4, TRA, TRB, B2M, albumin, HBB, HBA1, HBA2, HBG1, HBG2, HBD, HBEl, TTR, NR3C1, CD52, erythroid specific enhancer of the BCL11A gene, CBLB, TGFBR1, SERPINA1, HBV genomic DNA in infected cells, CEP290, DMD, CFTR, IL2RG, CS-1, or any combination thereof. In some embodiments, a genome editing complex can cleave double stranded DNA at a target site in order to insert a chimeric antigen receptor (CAR), alpha-L iduronidase (IDUA), iduronate-2-sulfatase (IDS), or Factor 9 (F9). Cells, such as hematopoietic stem cells (HSCs) and T cells, can be engineered ex vivo with the genome editing complex. Alternatively, genome editing complexes can be directly administered to a subject in need thereof.

### Compositions

In certain aspects, the polypeptides described herein may be present in a composition, e.g., a pharmaceutical composition comprising a pharmaceutically acceptable excipient. In certain aspects, the polypeptides are present in a therapeutically effective amount in the pharmaceutical composition. A therapeutically effective amount can be determined based on an observed effectiveness of the composition. A therapeutically effective amount can be determined using assays that measure the desired effect in a cell, e.g., in a reporter cell line in which expression of a reporter is modulated in response to the polypeptides of the present disclosure. The pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein. Suitable pharmaceutically acceptable or physiologically acceptable diluents, carriers or excipients include, but are not limited to, nuclease inhibitors, protease inhibitors, a suitable vehicle such as physiological saline solution or citrate buffered saline.

In certain embodiments, the composition may include (i) a polypeptide comprising at least three RUs as disclosed herein, wherein the polypeptide NBD is fused to the first binding member as disclosed herein; and (ii) a second binding member as disclosed herein. In certain embodiments, the polypeptide and the second binding member may be present in form of a heterodimer.

In certain embodiments, the composition may include (i) a polypeptide comprising at least three RUs as disclosed herein, wherein the polypeptide NBD is fused to the second binding member as disclosed herein; and (ii) a first binding member as disclosed herein. In certain embodiments, the polypeptide and the first binding member may be present in form of a heterodimer.

### Delivery

The positively charged polypeptides disclosed herein and compositions comprising the disclosed polypeptides can be delivered into a target cell by any suitable means, including, for example, by contacting the cell with the polypeptide. In certain aspects, the positively charged polypeptides can be delivered into cells in a particular tissue (e.g., a solid tumor) by injecting a composition comprising the positively charged polypeptide directly into the solid tumor.

In other aspects, administration involves systemic administration (e.g., intravenous, intraperitoneal, intramuscular, subdermal, or intracranial infusion), direct injection (e.g., intrathecal), or topical application, etc.

### Methods

The present invention provides methods for producing the disclosed polypeptides. In particular embodiments, the polypeptides may be produced in vitro using a cell line. In certain embodiments, the polypeptides may be produced in a cell-free in vitro transcription translation system.

The polypeptides may include certain tags, such as, purification tag, detection/imaging tags. Such tags may be attached to the polypeptides of the invention via a cleavable regions to facilitate removal of the tag after purification, for example.

The present invention also provides a method of introducing positively charged genome modifying proteins, with or without an agent associated with the positively charged proteins, into a cell. The method comprises contacting the positively charged polypeptide(s), or a positively charged polypeptide and an agent associated with the positively charged polypeptide (e.g., where the agent is negatively charged and associates with the positively charged polypeptide via electrostatic interaction) with the cell, e.g., under conditions sufficient to allow penetration of the positively charged polypeptide, or an agent associated with the positively charged polypeptide, into the cell, thereby introducing a the positively charged polypeptide, or an agent associated with the positively charged polypeptide, or both, into a cell. In certain aspects, introduction of the positively charged polypeptide may be assessed by assaying the cell for presence of a signal indicative of the entry or assaying for an effect of the positively charged polypeptide in the cell.

In certain embodiments, the contact is performed in vitro. In certain embodiments, the contact is performed in vivo, e.g., in the body of a subject, e.g., a human or other animal or ex vivo. In one in vivo embodiment, sufficient positively charged polypeptide is present in the cell to provide a detectable effect in the subject, e.g., a therapeutic effect. In one in vivo embodiment, sufficient positively charged polypeptide is present in the cell to allow imaging of one or more penetrated cells or tissues. In certain embodiments, the observed or detectable effect arises from cell penetration.

The desired modifications or mutations in a polypeptide may be accomplished using any techniques known in the art. Recombinant DNA techniques for introducing such changes in a protein sequence are well known in the art. In certain embodiments, the modifications are made by site-directed mutagenesis of the polynucleotide encoding the protein. Other techniques for introducing mutations are discussed in Molecular Cloning: A Laboratory Manual, 2nd Ed., ed. by Sambrook, Fritsch, and Maniatis (Cold Spring Harbor Laboratory Press: 1989); the treatise, Methods in Enzymology (Academic Press, Inc., N.Y.); Ausubel et al. Current Protocols in Molecular Biology (John Wiley & Sons, Inc., New York, 1999). The modified protein is expressed and tested. In certain embodiments, a series of variants is prepared, and each variant is tested to determine its biological activity and its stability. The variant chosen for subsequent use may be the most stable one, the most active one, or the one with the greatest overall combination of activity and stability. After a first set of variants is prepared an additional set of variants may be prepared based on what is learned from the first set. Variants are typically created and overexpressed using recombinant techniques known in the art.

The polypeptide provided herein may be modified to increase yield, half-life, activity of the polypeptide. Such modifications include PEGylation, glycosylation, lipidation, conjugation to Fc portion of human IgG, maltose binding proteins, albumin and the like. In certain aspects, the polypeptides (e.g., the NBDs, functional domains, conjugates thereof, and the like) provided herein may be fused to a peptide that enhances endosome degradation or lysis of the endosome to reduce sequestration of the polypeptides in the endosomes. In certain embodiments, the peptide is hemagglutinin 2 (HA2) peptide which is known to enhance endosome degradation.

A method of modulating expression of an endogenous gene in a cell is also provided. The method may include contacting the cell with the positively charged polypeptide as provided herein, wherein the polypeptide penetrates the cell membrane and wherein the NBD of the polypeptide binds to a target nucleic acid sequence present in the endogenous gene and the heterologous functional domain modulates expression of the endogenous gene. The nucleic acid may be a ribonucleic acid (RNA) or a deoxyribonucleic acid (DNA).

The functional domain may be a transcriptional activator and the target nucleic acid sequence is present in an expression control region of the gene, wherein the polypeptide increases expression of the gene. The transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta).

In other aspects, the functional domain is a transcriptional repressor and the target nucleic acid sequence is present in an expression control region of the gene, wherein the polypeptide decreases expression of the gene. The transcriptional repressor may be KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

The an endogenous gene may be a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEA1 gene, a TTR gene, a NR3C1 gene, a CD52 gene, an erythroid specific enhancer of the ECL11A gene, a CELE gene, a TGFER1 gene, a SERPINA1 gene, a HEV genomic DNA in infected cells, a CEP290 gene, a DMD gene, a CFTR gene, or an IL2RG gene.

The expression control region of the gene may include a promoter region of the gene.

The functional domain may be a nuclease comprising a cleavage domain or a half-cleavage domain and the endogenous gene is inactivated by cleavage.

In certain aspects, the polypeptide is a first polypeptide that binds to a first target nucleic acid sequence in the gene and comprises a half-cleavage domain and the method comprises introducing a second polypeptide that binds to a second target nucleic acid sequence in the gene and comprises a half-cleavage domain. The first target nucleic acid sequence and the second target sequence may be spaced apart in the gene and the two half-cleavage domains mediate a cleavage of the gene sequence at a location in between the first and second target nucleic acid sequences. The cleavage domain or the cleavage half domain may be FokI or BfiI, or a meganuclease.

The target gene may be any gene of interest, such as, those disclosed herein.

In certain aspects, a method of introducing an exogenous nucleic acid into a region of interest in the genome of a cell is provided. The method may include introducing into the cell a positively charged polypeptide comprising a NBD as disclosed herein, where the NBD of the polypeptide binds to the target nucleic acid sequence present adjacent the region of interest; and the exogenous nucleic acid, wherein the cleavage domain or the half-cleavage domain introduces a cleavage in the region of interest and wherein the exogenous nucleic acid in integrated into the cleaved region of interest by homologous recombination.

In certain aspects, introducing the genome modifying proteins into the cell comprises contacting the cell with the proteins in absence of a transfection agent, wherein the proteins penetrates the cell membrane. In certain aspects, introducing the polypeptide and the exogenous nucleic acid into the cell comprises contacting the cell with a composition comprising the polypeptide associated with the exogenous nucleic acid, wherein the polypeptide penetrates the cell membrane and transports the exogenous nucleic acid into the cell. The cell may be any cell of interest, such as, those disclosed herein and the introducing may be performed in vivo, ex vivo or in vitro. In certain aspects, the introducing comprises administering the polypeptide to a subject. The administering may comprise parenteral administration. The administering may comprise intravenous, intramuscular, intrathecal, or subcutaneous administration. The administering may comprise direct injection into a site in a subject. The administering may comprise direct injection into a tumor, e.g., a solid tumor.

A method of modulating expression of an endogenous gene in a cell is disclosed, the method may include introducing into the cell the first binding member and the second binding member or a heterodimer as provided herein, wherein at least one of the first and second binding members penetrates the cell membrane and wherein the NBD binds to a target nucleic acid sequence present in the endogenous gene and the heterologous functional domain modulates expression of the endogenous gene. The NBD and the functional domain may be fused to the first and the second binding members or vice versa.

In certain aspects, introducing into the cell the first and second binding members comprises contacting the cell with the first and second binding members. In certain aspects, introducing into the cell the first and second binding members comprises contacting the cell with the first binding member and introducing into the cell a nucleic acid encoding the second binding member. In certain aspects, introducing into the cell the first and second binding members comprises contacting the cell with the second binding member and introducing into the cell a nucleic acid encoding the first binding member. The nucleic acid encoding the first or second binding member may be RNA or DNA. The NBD and the functional domain may be fused to the first and the second binding members or vice versa.

In certain aspects, the functional domain is a nuclease comprising a cleavage domain or a half-cleavage domain and the endogenous gene is inactivated by cleavage and wherein the first binding member comprises a NBD that binds to a first target nucleic acid sequence in the gene and the second binding member comprises a half-cleavage domain and the method comprises introducing a second first binding member comprising a NBD that binds to a second target nucleic acid sequence in the gene and a second binding member comprising a half-cleavage domain. In certain aspects, the first target nucleic acid sequence and the second target sequence are spaced apart in the gene and the two half-cleavage domains mediate a cleavage of the gene sequence at a location in between the first and second target nucleic acid sequences.

A method of introducing an exogenous nucleic acid into a region of interest in the genome of a cell is also provided. The method comprises:

introducing into the cell: the first binding member and the second binding member as disclosed herein, and the exogenous nucleic acid; or introducing into the cell: the first binding member and the second binding member as disclosed herein, and the exogenous nucleic acid, wherein the NBD of the polypeptide binds to the target nucleic acid sequence present adjacent the region of interest, wherein the cleavage domain or the half-cleavage domain introduces a cleavage in the region of interest and wherein the exogenous nucleic acid in integrated into the cleaved region of interest by homologous recombination. The NBD and the functional domain may be fused to the first and the second binding members or vice versa.

In certain aspects, introducing the first binding member and the second biding member into the cell comprises contacting the cell with the first and second binding members in absence of a transfection agent, wherein the first and second binding members penetrate the cell membrane. In certain aspects, introducing the first and second binding members and the exogenous nucleic acid into the cell comprises contacting the cell with a composition comprising the first and second binding members associated with the exogenous nucleic acid, wherein the first and second binding members penetrate the cell membrane and transports the exogenous nucleic acid into the cell. Introducing may include administering the first and second binding members to a subject by e.g., parenteral administration. In certain aspects, the administering comprises intravenous, intramuscular, intrathecal, or subcutaneous administration. In certain aspects, the administering comprises direct injection into a site in a subject. In certain aspects, the administering comprises direct injection into a tumor.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for.

### Example 1: Cell Permeable Components of Genome Modifying Complex

The following components were synthesized and tested.

COMPONENT 1, #1: TL8188_Q31K_3x37A+

(SEQ ID NO: 168)
MAHHHHHHLATTHMGSSNSNNATMAPDRVRAVSHWSSGGSMASMT
GGQQMGGGAGKPIPNPLLGLDSTGAPKKKRKVGIHRGVPMVDLRTLGYSQQQQEKI
KPKVRSTVAQHHEALVGHGFTHAHIVALSQHPAALGTVAVKYQDMIAALPEATHEAIV
GVGKOWSGARALEALLTVAGELRGPPLQLDTGOLLKIAKRGGVTAVEAVHAWRNALT

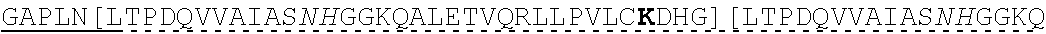

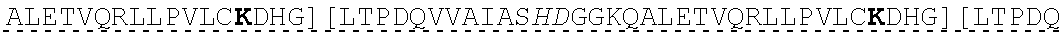

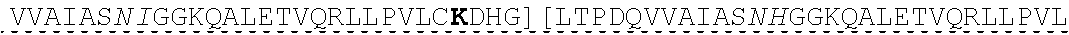

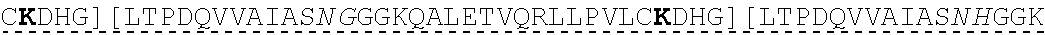

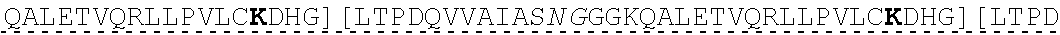

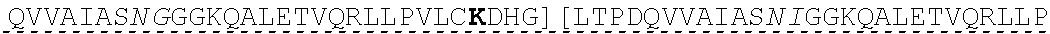

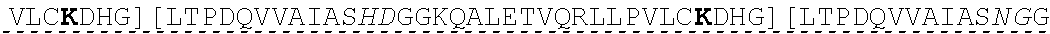

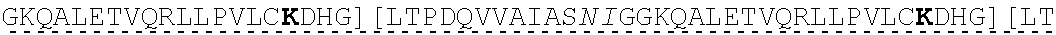

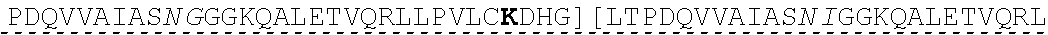

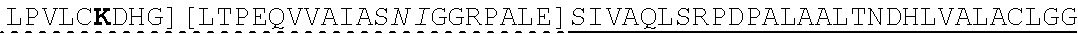

RPALDAVKKGLPHAPALIKRTNRRIPERTSHRVAGSGGGMDAKSLTAWSGKGSKGKGK
GKGSKDSKKHLKKLKKFLENLRRHLDRLKKHIKQLRKILKENPKDKRVKDVIDKSERS
VRIVKKVIKIFEKSVRKKEGGGGGMDAKSLTAWSGKGSKGKGKGKGSKDSKKHLKKL
KKFLENLRRHLDRLKKHIKQLRKILKENPKDKRVKDVIDKSERSVRIVKKVIKIFEKSVR
KKEGGGGGMDAKSLTAWSGKGSKGKGKGKGSKDSKKHLKKLKKFLENLRRHLDRLK
KHIKQLRKILKENPKDKRVKDVIDKSERSVRIVKKVIKIFEKSVRKKE

HHHHHH=6×-His Tag (SEQ ID NO:169); PDRVRAVSHWSS=SPOT tag (SEQ ID NO:170); MASMTGGQQMG=T7 tag (SEQ ID NO:171); GKPIPNPLLGLDST=V5 tag (SEQ ID NO:172); PKKKRKV=SV40 NLS (SEQ ID NO: 173); TALE N-cap region and TALE C-cap region are underlined.

The DBD contains 15 RUs, each comprising K at position 31 (indicated in bold). The DBD binds to the promote region of TIM3 gene. Each RU is in brackets [RU] and is underlined with discontinuous line. RVDs are italicized. N-cap and C-cap regions are underlined.

(SEQ ID NO: 164)
GGGMDAKSLTAWS = Uncharged linkers
(SEQ ID NO: 174)
GKGSKGKGKGKGSKDSKKHLKKLKKFLENLRRHLDRLKKHIKQLRKILK
ENPKDKRVKDVIDKSERSVRIVKKVIKIFEKSVRKKE = Charged 37A
COMPONENT 1, #2: TL8188_Q31K, E20R_3x37A++
(SEQ ID NO: 175)
MAHHHHHHLATTHMGSSNSNNATMAPDRVRAVSHWSSGGSMASMTG
GQQMGGGAGKPIPNPLLGLDSTGAPKKKRKVGIHRGVPMVDLRTLGYSQQQQEKIKPK
VRSTVAQHHEALVGHGFTHAHIVALSQHPAALGTVAVKYQDMIAALPEATHEAIVGVG
KQWSGARALEALLTVAGELRGPPLOLDTGQLLKIAKRGGVTAVEAVHAWRNALTGAP
LNLTPDQVVAIASNHGGKQALRTVQRLLPVLCKDHGLTPDQVVAIASNHGGKQALRTV
QRLLPVLCKDHGLTPDQVVAIASHDGGKQALRTVQRLLPVLCKDHGLTPDQVVAIASNI
GGKQALRTVQRLLPVLCKDHGLTPDQVVAIASNHGGKQALRTVQRLLPVLCKDHGLTP
DQVVAIASNGGGKQALRTVQRLLPVLCKDHGLTPDQVVAIASNHGGKQALRTVQRLLP
VLCKDHGLTPDQVVAIASNGGGKQALRTVQRLLPVLCKDHGLTPDQVVAIASNGGGK
QALRTVQRLLPVLCKDHGLTPDQVVAIASNIGGKQALRTVQRLLPVLCKDHGLTPDQV
VAIASHDGGKQALRTVQRLLPVLCKDHGLTPDQVVAIASNGGGKQALRTVQRLLPVLC
KDHGLTPDQVVAIASNIGGKQALRTVQRLLPVLCKDHGLTPDQVVAIASNGGGKQALR
TVQRLLPVLCKDHGLTPDQVVAIASNIGGKQALRTVQRLLPVLCKDHGLTPEQVVAIAS
NIGGRPALESIVAQLSRPDPALAALTNDHLVALACLGGRPALDAVKKGLPHAPALIKRT
NRRIPERTSHRVAGSGSKGKGKGKMDAKSLTAWSGKGSKGKGKGKGSKDSKKHLKKL
KKFLENLRRHLDRLKKHIKQLRKILKENPKDKRVKDVIDKSERSVRIVKKVIKIFEKSVR
KKEGGGSKGKGKGKMDAKSLTAWSGKGSKGKGKGKGSKDSKKHLKKLKKFLENLRR
HLDRLKKHIKQLRKILKENPKDKRVKDVIDKSERSVRIVKKVIKIFEKSVRKKEGGGSKG
KGKGKMDAKSLTAWSGKGSKGKGKGKGSKDSKKHLKKLKKFLENLRRHLDRLKKHI
KQLRKILKENPKDKRVKDVIDKSERSVRIVKKVIKIFEKSVRKKE
(SEQ ID NO: 169)
HHHHHH = His Tag
(SEQ ID NO: 170)
PDRVRAVSHWSS = SPOT tag;
(SEQ ID NO: 171)
MASMTGGQQMG = T7 tag;
(SEQ ID NO: 172)
GKPIPNPLLGLDST = V5 tag;
(SEQ ID NO: 173)
PKKKRKV = SV40 NLS;
TALE N-cap region and TALE C-cap region are underlined.

The DBD contains 15 RUs, each comprising K at position 31 (indicated in bold). The DBD binds to the promote region of TIM3 gene.

(SEQ ID NO: 164)
GGGMDAKSLTAWS = Uncharged linkers
(SEQ ID NO: 174)
GKGSKGKGKGKGSKDSKKHLKKLKKFLENLRRHLDRLKKHIKQLRKILK
ENPKDKRVKDVIDKSERSVRIVKKVIKIFEKSVRKKE =
Charged 37A

The DBD contains 15 RUs, each comprising the substitutions: E20R and Q31K.

(SEQ ID NO: 166)
GSKGKGKGKMDAKSLTAWS = Charged linkers
(SEQ ID NO: 174)
GKGSKGKGKGKGSKDSKKHLKKLKKFLENLRRHLDRLKKHIKQLRKILK
ENPKDKRVKDVIDKSERSVRIVKKVIKIFEKSVRKKE = Charged 37A
COMPONENT 2: 37B+_KRAB
(SEQ ID NO: 176)
MATTHNHHHHHHHHHPILGYWKIKGLVQPTRLLLEYLEEKYEEHLYERD
EGDKWRNKKFELGLEFPNLPYYIDGDVKLTQSMAIIRYIADKHNMLGGCPKERAEISML
EGAVLDIRYGVSRIAYSKDFETLKVDFLSKLPEMLKMFEDRLCHKTYLNGDHVTHPDF
MLYDALDVVLYMDPMCLDAFPKLVCFKKRIEAIPQIDKYLKSSKYIAWPLQGWQATFG
GGDHPPKSDLVPREPTTLEVLFQGPDAYPYDVPDYAGAPKKKRKVGAKKDKKLDKLL
DKLEKILQKATKIIDKANKLLEKLRRSKRKKPKVVKTYVELLKRHEKAVKELLEIAKTH
AKKVEGKGSKGKGKGKMDAKSLTAWSRTLVTFKDVFVDFTREEWKLLDTAQQIVYR
NVMLENYKNLVSLGYQLTKPDVILRLEKGEEP
(SEQ ID NO: 177)
HHHHHHHHH = 9X-His tag
(SEQ ID NO: 178)
PILGYWKIKGLVQPTRLLLEYLEEKYEEHLYERDEGDKWRNKKFELGLE
FPNLPYYIDGDVKLTQSMAIIRYIADKHNMLGGCPKERAEISMLEGAVLDIRYGVSRIAY
SKDFETLKVDFLSKLPEMLKMFEDRLCHKTYLNGDHVTHPDFMLYDALDVVLYMDPM
CLDAFPKLVCFKKRIEAIPQIDKYLKSSKYIAWPLQGWQATFGGGDHPPK = GST
(SEQ ID NO: 179)
LEVLFQGP = HRV-3C protease cleavage site
(SEQ ID NO: 180)
YDVPDYA = HA tag
(SEQ ID NO: 173)
PKKKRKV = NLS
(SEQ ID NO: 181)
KKDKKLDKLLDKLEKILQKATKIIDKANKLLEKLRRSKRKKPKVVKTYV
ELLKRHEKAVKELLEIAKTHAKKVE = Charged 37B
(SEQ ID NO: 140)
GKGSKGKGKGK = Charged 37B linker
(SEQ ID NO: 182)
MDAKSLTAWSRTLVTFKDVFVDFTREEWKLLDTAQQIVYRNVMLENYK
NLVSLGYQLTKPDVILRLEKGEEP = KRAB

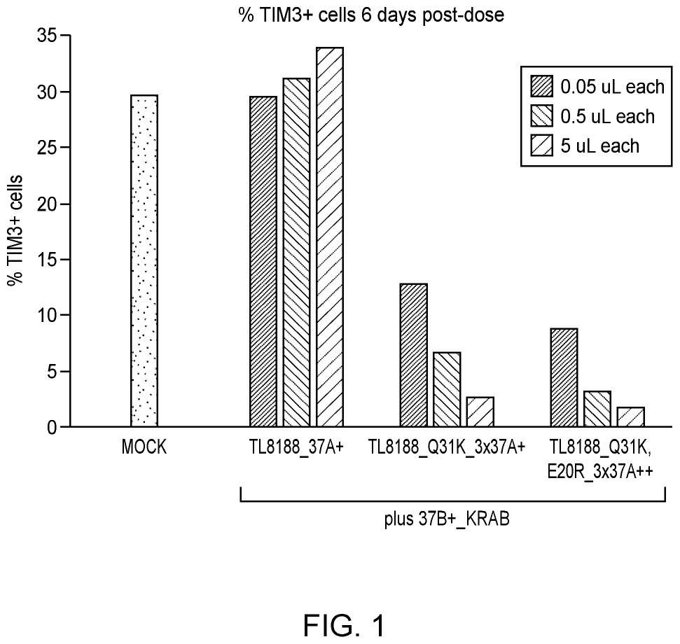

As shown in FIG. 1, introduction of component 1 (TL8188_37A+): a DBD comprising RUs (that do not include the substitution Q31K or E20R and target TIM3 gene) fused to positively charged 37A and component 2 (37B+_KRAB): a positively charged 37B fused to KRAB did not result in significant suppression of TIM3 expression in the treated cells. In contrast, introduction of component 1 (TL8188_Q31K_3X37A+): a DBD comprising RUs (that include the substitution Q31K and target TIM3 gene) fused to three copies of positively charged 37A and component 2 (37B+_KRAB) result in significant suppression of TIM3 expression in the treated cells. Introduction of component 1 (TL8188_Q31K,E20R_3X37A++): a DBD comprising RUs (that include the substitutions Q31K, E20R and target TIM3 gene) fused to three copies of positively charged 37A via charged linkers and component 2 (37B+_KRAB) result in significant suppression of TIM3 expression in the treated cells. The suppression was dose-dependent.

Protein preparations of TL8188_37A+, TL8188_Q31K_3x37A+, TL8188_Q31K,E20R_3x37A++ and 37B+_KRAB were made using the 1-Step Human Coupled IVT Kit (Thermo Fisher Scientific). TL8188_37A+, TL8188_Q31K_3x37A+, TL8188_Q31K,E20R_3x37A++ were each mixed with an equal volume of 37B+_KRAB. Primary human CD4+ T cells that had been stimulated for 24 h with CD3/CD28 Dynabeads (Thermo Fisher Scientific) were centrifuged at 430 g for 5 min and resuspended in serum-free media (RPMI, 1% penicillin/streptomycin, 50 U/mL IL2) at a concentration of 5.55×10≢cells/mL. 90 uL cells (500,000) were added to 0.1 uL, 1 uL and 10 uL of each of the IVT mixtures (each first supplemented to 10 uL with media). Cells were incubated with the IVT preparations for 4 hours at 37° C. and subsequently transferred to 720 uL pre-warmed full media (as above, but containing 10% fetal bovine serum). After 6 days, cells were analyzed for TIM3 expression by FACS using Brilliant Violet 421™ anti-human CD366 (TIM3) antibody (clone F38-2E2, BioLegend).

For reasons of completeness, certain aspects of the polypeptides, composition, and methods of the present disclosure are set out in the following numbered clauses:
1. A polypeptide comprising a nucleic acid-binding domain (NBD) comprising: at least three repeat units (RUs) comprising a 33-36 amino acid long sequence having at least 80% sequence identity to the amino acid sequence:
   LTPDQ VVAIA SX¹²X¹³GG KQALE TVQRL LPVLC QDHG (SEQ ID NO:1), or
   having the sequence of SEQ ID NO:1 with one or more conservative amino acid substitutions thereto; and comprising one or both of the following amino acid substitutions relative to SEQ ID NO:1: E20R/K/H and Q31K/R/H,
   wherein X¹²X¹³ is HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means X₁₃ is absent,
   wherein when the RUs comprise the substitution Q31K/R/H,
      X¹²X¹³ is not NK, YK or HN,
      the amino acid at position 32 is not P,
      the RUs further comprise the substitution E20R/K/H, and/or
      the RUs are 33-34 amino acid long;
   wherein when the RUs comprise the substitution E20R/K/H,
      X₁₂X₁₃ is not HD, HN, KG, KI, or
      the amino acid at position 32 is not P,
      the RUs further comprise the substitution Q31K/R/H, and/or
      the RUs are 33-34 amino acid long.
2. The polypeptide according to clause 1, wherein the RUs comprise the substitution Q31K/R/H and X¹²X¹³ is not NK, YK or HN.
3. The polypeptide according to clause 1 or 2, wherein the RUs comprise the substitution Q31K/R/H and the amino acid at position 32 is not P.
4. The polypeptide according to any one of clauses 1-3, wherein the RUs further comprise the substitution E20R/K/H.
5. The polypeptide according to any one of clauses 1-4, wherein the RUs are 33-34 amino acid long.
6. The polypeptide according to any one of clauses 1-5, wherein the RUs comprise the substitutions Q31K/R/H and E20R/K/H.
7. The polypeptide according to any one of clauses 1-6, wherein the RUs comprise the substitutions Q31K and E20R or Q31K and E20K or Q31R and E20R.
8. The polypeptide according to any one of clauses 1-7, wherein the at least three RUs comprise a 33-36 amino acid long sequence having at least 90% sequence identity to the amino acid sequence of SEQ ID NO:1.
9. The polypeptide according to any one of clauses 1-8, wherein the at least three RUs comprise the amino acid sequence:

(SEQ ID NO: 158)
LTPDQ VVAIA SX¹²X¹³GG KQALR/K/H TVQRL LPVLC QDHG;
(SEQ ID NO: 159)
LTPDQ VVAIA SX¹²X¹³GG KQALE TVQRL LPVLC K/R/HDHG;
(SEQ ID NO: 160)
LTPDQ VVAIA SX¹²X¹³GG KQALR/K/H TVQRL LPVLC
K/R/HDHG;
(SEQ ID NO: 161)
LTPDQ VVAIA SX¹²X¹³GG KQALR TVQRL LPVLC KDHG;
or
(SEQ ID NO: 183)
LTPDQ VVAIA SX¹²X¹³GG KQALE TVQRL LPVLC KDHG.

10. The polypeptide according to any one of clauses 1-9, comprising ten to twenty of the RUs or twelve to twenty of the RUs.
11. The polypeptide of any one of clauses 1-10, fused to a first binding member of a heterodimer or to a second binding member of a heterodimer, wherein the first binding member binds to a second binding member of the heterodimer, wherein the first binding member comprises an amino acid sequence at least 75% identical to the amino acid sequence of SEQ ID NO:2 and the second binding member comprises an amino acid sequence at least 75% identical to the amino acid sequence of SEQ ID NO:3, wherein the N-terminus or the C-terminus of the NBD is fused to the first or the second binding member.
12. The polypeptide of clause 11, wherein the C-terminus of the NBD is fused to the N-terminus of the first or the second binding member.
13. The polypeptide of clause 11 or 12, wherein the polypeptide is fused to the first binding member and wherein the amino acid sequence of the first binding member comprises at least one of the following substitutions relative to the amino acid sequence of SEQ ID NO:2: D3K/R/H; E4K/R/H; T11K/R/H; D24K/R/H; D32K/R/H; S35K/R/H; E39K/R/H; D40K/R/H; E41K/R/H; D45K/R/H; D48K/R/H; L49K/R/H; T59K/R/H; and D66K/R/H.
14. The polypeptide of clause 13, wherein the first binding member comprises at least three of the substitutions.
15. The polypeptide of clause 13, wherein the first binding member comprises at least five of the substitutions.
16. The polypeptide of clause 13, wherein the first binding member comprises at least eight of the substitutions.
17. The polypeptide of any one of clauses 11-16, wherein the first binding member comprises the amino acid sequence:

(SEQ ID NO: 8)
DSKKHLKKLKKFLENLRRHLDRLKKHIKQLRKILKENPKDKRVKDVIDK
SERSVRIVKKVIKIFEKSVRKKE.

18. The polypeptide of any one of clauses 11-17, wherein the first binding member comprises a positively charged tag sequence fused to the N-terminus or C-terminus of the first binding member.
19. The polypeptide of clause 18, wherein the positively charged tag sequence is fused to the N-terminus of the first binding member.
20. The polypeptide of clause 18 or 19, wherein the positively charged tag sequence comprises the amino acid sequence: GKGSKGKGKGKGSK (SEQ ID NO:141).
21. The polypeptide of any one of clauses 11-20, wherein the NBD is fused to the first or the second binding member via a linker.
22. The polypeptide of clause 21, wherein the linker is a positively charged linker.
23. The polypeptide of clause 22, wherein the positively charged linker comprises the amino acid sequence: GSKGKGKGKMDAKSLTAWS (SEQ ID NO:166).
24. The polypeptide of any one of clauses 11-23, wherein NBD is fused to multiple copies of the first or the second binding member.
25. The polypeptide of clause 24, wherein the NBD is fused to two or three copies of the first binding member.
26. The polypeptide of clause 25, wherein the linker connects the multiple copies of the first binding member to each other.
27. The polypeptide of clause 11 or 12, wherein the NBD is fused to the second binding member.
28. The polypeptide of clause 27, wherein the second binding member comprises at least one of the following substitutions relative to the amino acid sequence of SEQ ID NO:3: D2K/R/H; D3K/R/H; E5K/R/H; T12K/R/H; T19K/R/H; D26K/R/H; E38K/R/H; D41K/R/H; E46K/R/H; E56K/R/H; E61K/R/H; T68K/R/H; and E74K/R/H.
29. The polypeptide of clause 28, wherein the second binding member comprises the amino acid sequence:

(SEQ ID NO: 181)
KKDKKLDKLLDKLEKILQKATKIIDKANKLLEKLRRSKRKKPKVVKTYV
ELLKRHEKAVKELLEIAKTHAKKVE.

30. The polypeptide of any one of clauses 1-29, wherein the NBD comprises an N-cap domain comprising the amino acid sequence:

(SEQ ID NO: 184)
GIHRGVPMVDLRTLGYSQQQQEKIKPKVRSTVAQHHEALVGHGFTHAHI
VALSQHPAALGTVAVKYQDMIAALPEATHEAIVGVGKQWSGARALEALL
TVAGELRGPPLQLDTGQLLKIAKRGGVTAVEAVHAWRNALTGAPLN.

31. The polypeptide of any one of clauses 1-30, wherein the NBD comprises a C-cap domain comprising the amino acid sequence:

(SEQ ID NO: 185)
SIVAQLSRPDPALAALTNDHLVALACLGGRPALDAVKKGLPHAPALIKR
TNRRIPERTSHRVAGS.

32. The polypeptide of any one of clauses 1-31, wherein the polypeptide comprises a positively charged purification tag.
33. The polypeptide of clause 32, wherein the positively charged purification tag is a poly-histidine tag.
34. The polypeptide of any one of clauses 1-33, wherein the polypeptide comprises a positively charged nuclear localization sequence.
35. The polypeptide of clause 34, wherein the nuclear localization sequence comprises the sequence PKKKRKV (SEQ ID NO:173).
36. The polypeptide of any one of clauses 1-35, wherein the NBD of the polypeptide binds to a region of the TIM3 gene, PD-L1 gene, PDCD1 gene, CTLA4 gene, or LAG3 gene.
37. The polypeptide of any one of clauses 1-35, wherein the NBD of the polypeptide binds to a promoter region of a gene.
38. The polypeptide of clause 37, wherein the gene is TIM3 gene, PD-L1 gene, PDCD1 gene, CTLA4 gene, or LAG3 gene.
39. The polypeptide of any one of clauses 1-38, wherein the polypeptide is produced in vitro.
40. The polypeptide of any one of clauses 1-38, wherein the polypeptide is produced in a cell-free in vitro transcription translation system.
41. A second binding member of a heterodimer, wherein the second binding member comprises an amino acid sequence at least 75% identical to the amino acid sequence of SEQ ID NO:3 and comprises at least one of the following substitutions relative to the amino acid sequence of SEQ ID NO:3: D2K/R/H; D3K/R/H; E5K/R/H; T12K/R/H; T19K/R/H; D26K/R/H; E38K/R/H; D41K/R/H; E46K/R/H; E56K/R/H; E61K/R/H; T68K/R/H; and E74K/R/H and wherein the second binding member binds to a first binding member of the heterodimer, wherein the first binding member comprises an amino acid sequence at least 75% identical to the amino acid sequence of SEQ ID NO:2 and wherein the second binding member is fused to a nuclear localization sequence (NLS).
42. The second binding member of clause 41, wherein the NLS is positively charged.
43. The second binding member of clause 42, wherein the NLS comprises the sequence PKKKRKV (SEQ ID NO:173).
44. The second binding member of any one of clauses 41-43, comprising at least three of the substitutions.
45. The second binding member of any one of clauses 41-43, comprising at least five of the substitutions.
46. The second binding member of any one of clauses 41-43, comprising at least seven of the substitutions.
47. The second binding member of any one of clauses 41-46, comprising the amino acid sequence:

(SEQ ID NO: 181)
KKDKKLDKLLDKLEKILQKATKIIDKANKLLEKLRRSKRKKPKVVKTYV
ELLKRHEKAVKELLEIAKTHAKKVE.

48. The second binding member of any one of clauses 41-47, wherein the second binding member is fused to a functional domain, wherein the NLS is fused to the N-terminus of the second binding member and the functional domain is fused to the C-terminus of the second binding member.
49. The second binding member of clause 48, wherein the second binding member is fused to the functional domain via a linker sequence.
50. The second binding member of clause 49, wherein the linker sequence is positively charged.
51. The second binding member of clause 50, wherein the linker sequence comprises the amino acid sequence: GKGSKGKGKGK (SEQ ID NO:140).
52. The second binding member of any one of clauses 48-51, wherein the functional domain comprises an enzyme, a transcriptional activator, a transcriptional repressor, or a DNA nucleotide modifier.
53. The second binding member of clause 52, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.
54. The second binding member of any one of clauses 41-53, wherein the second binding member is produced in vitro.
55. The second binding member of any one of clauses 41-53, wherein the second binding member is produced in a cell-free in vitro transcription translation system.
56. A composition comprising: (i) a polypeptide according to any one of clauses 13-26 or 31-40, wherein the polypeptide NBD is fused to the first binding member; and (ii) a second binding member according to any one of clauses 41-55.
57. A first binding member of a heterodimer, wherein the first binding member comprises an amino acid sequence at least 75% identical to the amino acid sequence of SEQ ID NO:2 and comprises at least one of the following substitutions relative to the amino acid sequence of SEQ ID NO:2: D3K/R/H; E4K/R/H; T11K/R/H; D24K/R/H; D32K/R/H; S35K/R/H; E39K/R/H; D40K/R/H; E41K/R/H; D45K/R/H; D48K/R/H; L49K/R/H; T59K/R/H; and D66K/R/H and wherein the first binding member binds to a second binding member of the heterodimer, wherein the second binding member comprises an amino acid sequence at least 75% identical to the amino acid sequence of SEQ ID NO:3 and wherein the first binding member is fused to a nuclear localization sequence (NLS).
58. The first binding member of clause 57, comprising at least three of the substitutions.
59. The first binding member of clause 57, comprising at least five of the substitutions.
60. The first binding member of clause 57, comprising at least eight of the substitutions.
61. The first binding member of any one of clauses 57-60, wherein the NLS is positively charged.
62. The first binding member of clause 61, wherein the NLS comprises the sequence

(SEQ ID NO: 173)
PKKKRKV.

63. The first binding member of any one of clauses 57-62, fused to a functional domain.
64. The first binding member of 63, wherein the functional domain is fused to the C-terminus of the first binding member and the NLS is fused to the N-terminus of the first binding member.
65. The first binding member of any one of clauses 63-64, wherein the first binding member is fused to the functional domain via a linker sequence.
66. The first binding member of clause 65, wherein the linker sequence is positively charged.
67. The first binding member of clause 66, wherein the linker sequence comprises the amino acid sequence: GKGSKGKGKGKMDAKSLTAWS (SEQ ID NO:162).
68. The first binding member of any one of clauses 63-67, wherein the functional domain comprises an enzyme, a transcriptional activator, a transcriptional repressor, or a DNA nucleotide modifier.
69. The first binding member of clause 68, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.
70. The first binding member of any one of clauses 57-69, wherein the first binding member is produced in vitro.
71. The first binding member of any one of clauses 57-69, wherein the first binding member is produced in a cell-free in vitro transcription translation system.
72. A composition comprising: (i) a polypeptide according to any one of clauses 27-40, wherein the polypeptide NBD is fused to the second binding member; and (ii) a first binding member according to any one of clauses 57-71.
73. A nucleic acid encoding the polypeptide of any one of clauses 1-40.
74. A nucleic acid encoding the second binding member of any one of clauses 41-55.
75. A nucleic acid encoding the first binding member of any one of clauses 57-71.
76. A method of modulating expression of an endogenous gene in a cell, the method comprising:
   contacting the cell with:
      (i) a polypeptide according to any one of clauses 13-26 or 31-40, wherein the polypeptide NBD is fused to the first binding member; and a second binding member according to any one of clauses 41-55;
      (ii) a polypeptide according to any one of clauses 27-40, wherein the polypeptide NBD is fused to the second binding member; and a first binding member according to any one of clauses 57-71,
      (iii) the composition of clause 56; or
      (iv) the composition of clause 72,
         wherein the polypeptide and the second binding member or the polypeptide and the first binding member penetrate the cell membrane and wherein the NBD of the polypeptide binds to a target nucleic acid sequence present in the endogenous gene and the heterologous functional domain modulates expression of the endogenous gene.
77. The method of clause 76, wherein the target nucleic acid is genomic DNA.
78. The method of clause 76 or 77, wherein the functional domain is a transcriptional activator and the target nucleic acid sequence is present in an expression control region of the gene, wherein the polypeptide increases expression of the gene.
79. The method of clause 78, wherein the transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta).
80. The method of clause 76 or 77, wherein the functional domain is a transcriptional repressor and the target nucleic acid sequence is present in an expression control region of the gene, wherein the polypeptide decreases expression of the gene.
81. The method of clause 80, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.
82. The method of any of clauses 76-81, wherein the gene is a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEA1 gene, a TTR gene, a NR3C1 gene, a CD52 gene, an erythroid specific enhancer of the BCL11A gene, a CELE gene, a TGFER1 gene, a SERPINA1 gene, a HEV genomic DNA in infected cells, a CEP290 gene, a DMD gene, a CFTR gene, or an IL2RG gene.
83. The method of any of clauses 80-82, wherein the expression control region of the gene comprises a promoter region of the gene.
84. The method of any of clauses 80-83, wherein the cell is an animal cell or plant cell.
85. The method of any of clauses 80-84, wherein the cell is a human cell.
86. The method of any of clauses 80-83, wherein the cell is an ex vivo cell.
87. The method of any of clauses 80-86, wherein the administering comprises parenteral administration.
88. The method of any of clauses 80-86, wherein the administering comprises intravenous, intramuscular, intrathecal, or subcutaneous administration.
89. The method of any of clauses 80-86, wherein the administering comprises direct injection into a site in a subject.
90. The method of any of clauses 80-86, wherein the administering comprises direct injection into a tumor.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A polypeptide comprising a nucleic acid-binding domain (NBD) comprising:
at least three repeat units (RUs) comprising a 33-36 amino acid long sequence having at least 80% sequence identity to the amino acid sequence:
LTPDQ VVAIA SX¹²X¹³GG KQALE TVQRL LPVLC QDHG (SEQ ID NO:1), or
having the sequence of SEQ ID NO:1 with one or more conservative amino acid substitutions thereto; and comprising one or both of the following amino acid substitutions relative to SEQ ID NO:1: E20R/K/H and Q31K/R/H,
wherein X¹²X¹³ is HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means X₁₃ is absent,
wherein when the RUs comprise the substitution Q31K/R/H,
X¹²X¹³ is not NK, YK or HN,
the amino acid at position 32 is not P,
the RUs further comprise the substitution E20R/K/H, and/or
the RUs are 33-34 amino acid long;
wherein when the RUs comprise the substitution E20R/K/H,
X₁₂X₁₃ is not HD, HN, KG, KI, or
the amino acid at position 32 is not P,
the RUs further comprise the substitution Q31K/R/H, and/or
the RUs are 33-34 amino acid long.

2. The polypeptide according to claim 1, wherein the RUs comprise the substitution Q31K/R/H and X¹²X¹³ is not NK, YK or HN.

3. The polypeptide according to claim 1 or 2, wherein the RUs comprise the substitution Q31K/R/H and the amino acid at position 32 is not P.

4. The polypeptide according to claim 1 or 2, wherein the RUs further comprise the substitution E20R/K/H.

5. The polypeptide according to claim 1 or 2, wherein the RUs are 33-34 amino acid long.

6. The polypeptide according to claim 1 or 2, wherein the RUs comprise the substitutions Q31K/R/H and E20R/K/H.

7. The polypeptide according to claim 1 or 2, wherein the RUs comprise the substitutions Q31K and E20R or Q31K and E20K or Q31R and E20R.

8. The polypeptide according to claim 1 or 2, wherein the at least three RUs comprise a 33-36 amino acid long sequence having at least 90% sequence identity to the amino acid sequence of SEQ ID NO:1.

9. The polypeptide according to claim 1 or 2, wherein the at least three RUs comprise the amino acid sequence:
(SEQ ID NO: 158)
LTPDQ VVAIA SX¹²X¹³GG KQALR/K/H TVQRL LPVLC QDHG;
(SEQ ID NO: 159)
LTPDQ VVAIA SX¹²X¹³GG KQALE TVQRL LPVLC K/R/HDHG;
(SEQ ID NO: 160)
LTPDQ VVAIA SX¹²X¹³GG KQALR/K/H TVQRL LPVLC
K/R/HDHG;
(SEQ ID NO: 161)
LTPDQ VVAIA SX¹²X¹³GG KQALR TVQRL LPVLC KDHG;
or
(SEQ ID NO: 183)
LTPDQ VVAIA SX¹²X¹³GG KQALE TVQRL LPVLC KDHG.

10. The polypeptide according to claim 1 or 2, comprising ten to twenty of the RUs or twelve to twenty of the RUs.

11. The polypeptide according to claim 1 or 2, fused to a first binding member of a heterodimer or to a second binding member of a heterodimer, wherein the first binding member binds to a second binding member of the heterodimer, wherein the first binding member comprises an amino acid sequence at least 75% identical to the amino acid sequence of SEQ ID NO:2 and the second binding member comprises an amino acid sequence at least 75% identical to the amino acid sequence of SEQ ID NO:3, wherein the N-terminus or the C-terminus of the NBD is fused to the first or the second binding member.

12. The polypeptide of claim 11, wherein the C-terminus of the NBD is fused to the N-terminus of the first or the second binding member.

13. The polypeptide of claim 12, wherein the polypeptide is fused to the first binding member and wherein the amino acid sequence of the first binding member comprises at least one of the following substitutions relative to the amino acid sequence of SEQ ID NO:2: D3K/R/H; E4K/R/H; T11K/R/H; D24K/R/H; D32K/R/H; S35K/R/H; E39K/R/H; D40K/R/H; E41K/R/H; D45K/R/H; D48K/R/H; L49K/R/H; T59K/R/H; and D66K/R/H.

14. The polypeptide of claim 13, wherein the first binding member comprises at least three of the substitutions.

15. The polypeptide of claim 13, wherein the first binding member comprises at least five of the substitutions.

16. The polypeptide of claim 13, wherein the first binding member comprises at least eight of the substitutions.

17. The polypeptide of claim 11, wherein the first binding member comprises the amino acid sequence:
(SEQ ID NO: 8)
DSKKHLKKLKKFLENLRRHLDRLKKHIKQLRKILKENPKDKRVKDVIDK
SERSVRIVKKVIKIFEKSVRKKE.

18. The polypeptide of claim 11, wherein the first binding member comprises a positively charged tag sequence fused to the N-terminus or C-terminus of the first binding member.

19. The polypeptide of claim 18, wherein the positively charged tag sequence is fused to the N-terminus of the first binding member.

20. The polypeptide of claim 18 or 19, wherein the positively charged tag sequence comprises the amino acid sequence: GKGSKGKGKGKGSK (SEQ ID NO:141).

21. The polypeptide of claim 11, wherein the NBD is fused to the first or the second binding member via a linker.

22. The polypeptide of claim 21, wherein the linker is a positively charged linker.

23. The polypeptide of claim 22, wherein the positively charged linker comprises the amino acid sequence: GSKGKGKGKMDAKSLTAWS (SEQ ID NO:166).

24. The polypeptide of claim 11, wherein NBD is fused to multiple copies of the first or the second binding member.

25. The polypeptide of claim 24, wherein the NBD is fused to two or three copies of the first binding member.

26. The polypeptide of claim 25, wherein the linker connects the multiple copies of the first binding member to each other.

27. The polypeptide of claim 11, wherein the NBD is fused to the second binding member.

28. The polypeptide of claim 27, wherein the second binding member comprises at least one of the following substitutions relative to the amino acid sequence of SEQ ID NO:3: D2K/R/H; D3K/R/H; E5K/R/H; T12K/R/H; T19K/R/H; D26K/R/H; E38K/R/H; D41K/R/H; E46K/R/H; E56K/R/H; E61K/R/H; T68K/R/H; and E74K/R/H.

29. The polypeptide of claim 28, wherein the second binding member comprises the amino acid sequence:
(SEQ ID NO: 181)
KKDKKLDKLLDKLEKILQKATKIIDKANKLLEKLRRSKRKKPKVVKTYV
ELLKRHEKAVKELLEIAKTHAKKVE.

30. The polypeptide of claim 1 or 2, wherein the NBD comprises an N-cap domain comprising the amino acid sequence:
(SEQ ID NO: 184)
GIHRGVPMVDLRTLGYSQQQQEKIKPKVRSTVAQHHEALVGHGFTHAHI
VALSQHPAALGTVAVKYQDMIAALPEATHEAIVGVGKQWSGARALEALL
TVAGELRGPPLQLDTGQLLKIAKRGGVTAVEAVHAWRNALTGAPLN.

31. The polypeptide of claim 1 or 2, wherein the NBD comprises a C-cap domain comprising the amino acid sequence:
(SEQ ID NO: 185)
SIVAQLSRPDPALAALTNDHLVALACLGGRPALDAVKKGLPHAPALIKR
TNRRIPERTSHRVAGS.

32. The polypeptide of claim 1, wherein the polypeptide comprises a positively charged purification tag.

33. The polypeptide of claim 32, wherein the positively charged purification tag is a poly-histidine tag.

34. The polypeptide of claim 1, wherein the polypeptide comprises a positively charged nuclear localization sequence.

35. The polypeptide of claim 34, wherein the nuclear localization sequence comprises the sequence PKKKRKV (SEQ ID NO:173).

36. The polypeptide of claim 1, wherein the NBD of the polypeptide binds to a region of the TIM3 gene, PD-L1 gene, PDCD1 gene, CTLA4 gene, or LAG3 gene.

37. The polypeptide of claim 1, wherein the NBD of the polypeptide binds to a promoter region of a gene.

38. The polypeptide of claim 37, wherein the gene is TIM3 gene, PD-L1 gene, PDCD1 gene, CTLA4 gene, or LAG3 gene.

39. The polypeptide of claim 1, wherein the polypeptide is produced in vitro.

40. The polypeptide of claim 1, wherein the polypeptide is produced in a cell-free in vitro transcription translation system.

41. A second binding member of a heterodimer, wherein the second binding member comprises an amino acid sequence at least 75% identical to the amino acid sequence of SEQ ID NO:3 and comprises at least one of the following substitutions relative to the amino acid sequence of SEQ ID NO:3: D2K/R/H; D3K/R/H; E5K/R/H; T12K/R/H; T19K/R/H; D26K/R/H; E38K/R/H; D41K/R/H; E46K/R/H; E56K/R/H; E61K/R/H; T68K/R/H; and E74K/R/H and wherein the second binding member binds to a first binding member of the heterodimer, wherein the first binding member comprises an amino acid sequence at least 75% identical to the amino acid sequence of SEQ ID NO:2 and wherein the second binding member is fused to a nuclear localization sequence (NLS).

42. The second binding member of claim 41, wherein the NLS is positively charged.

43. The second binding member of claim 42, wherein the NLS comprises the sequence
(SEQ ID NO: 173)
PKKKRKV.

44. The second binding member of claim 41, comprising at least three of the substitutions.

45. The second binding member of claim 41, comprising at least five of the substitutions.

46. The second binding member of claim 41, comprising at least seven of the substitutions.

47. The second binding member of claim 41, comprising the amino acid sequence:
(SEQ ID NO: 181)
KKDKKLDKLLDKLEKILQKATKIIDKANKLLEKLRRSKRKKPKVVKTYV
ELLKRHEKAVKELLEIAKTHAKKVE.

48. The second binding member of claim 41, wherein the second binding member is fused to a functional domain, wherein the NLS is fused to the N-terminus of the second binding member and the functional domain is fused to the C-terminus of the second binding member.

49. The second binding member of claim 48, wherein the second binding member is fused to the functional domain via a linker sequence.

50. The second binding member of claim 49, wherein the linker sequence is positively charged.

51. The second binding member of claim 50, wherein the linker sequence comprises the amino acid sequence: GKGSKGKGKGK (SEQ ID NO:140).

52. The second binding member of claim 48, wherein the functional domain comprises an enzyme, a transcriptional activator, a transcriptional repressor, or a DNA nucleotide modifier.

53. The second binding member of claim 52, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

54. The second binding member of claim 41, wherein the second binding member is produced in vitro.

55. The second binding member of claim 41, wherein the second binding member is produced in a cell-free in vitro transcription translation system.

56. A composition comprising: (i) a polypeptide according to any one of claims 13-26 or 31-40, wherein the polypeptide NBD is fused to the first binding member; and (ii) a second binding member according to any one of claims 41-55.

57. A first binding member of a heterodimer, wherein the first binding member comprises an amino acid sequence at least 75% identical to the amino acid sequence of SEQ ID NO:2 and comprises at least one of the following substitutions relative to the amino acid sequence of SEQ ID NO:2: D3K/R/H; E4K/R/H; T11K/R/H; D24K/R/H; D32K/R/H; S35K/R/H; E39K/R/H; D40K/R/H; E41K/R/H; D45K/R/H; D48K/R/H; L49K/R/H; T59K/R/H; and D66K/R/H and wherein the first binding member binds to a second binding member of the heterodimer, wherein the second binding member comprises an amino acid sequence at least 75% identical to the amino acid sequence of SEQ ID NO:3 and wherein the first binding member is fused to a nuclear localization sequence (NLS).

58. The first binding member of claim 57, comprising at least three of the substitutions.

59. The first binding member of claim 57, comprising at least five of the substitutions.

60. The first binding member of claim 57, comprising at least eight of the substitutions.

61. The first binding member of any one of claims 57-60, wherein the NLS is positively charged.

62. The first binding member of claim 61, wherein the NLS comprises the sequence
(SEQ ID NO: 173)
PKKKRKV.

63. The first binding member of any one of claims 57-62, fused to a functional domain.

64. The first binding member of 63, wherein the functional domain is fused to the C-terminus of the first binding member and the NLS is fused to the N-terminus of the first binding member.

65. The first binding member of any one of claims 63-64, wherein the first binding member is fused to the functional domain via a linker sequence.

66. The first binding member of claim 65, wherein the linker sequence is positively charged.

67. The first binding member of claim 66, wherein the linker sequence comprises the amino acid sequence: GKGSKGKGKGKMDAKSLTAWS (SEQ ID NO:162).

68. The first binding member of any one of claims 63-67, wherein the functional domain comprises an enzyme, a transcriptional activator, a transcriptional repressor, or a DNA nucleotide modifier.

69. The first binding member of claim 68, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

70. The first binding member of any one of claims 57-69, wherein the first binding member is produced in vitro.

71. The first binding member of any one of claims 57-69, wherein the first binding member is produced in a cell-free in vitro transcription translation system.

72. A composition comprising: (i) a polypeptide according to any one of claims 27-40, wherein the polypeptide NBD is fused to the second binding member; and (ii) a first binding member according to any one of claims 57-71.

73. A nucleic acid encoding the polypeptide of any one of claims 1-40.

74. A nucleic acid encoding the second binding member of any one of claims 41-55.

75. A nucleic acid encoding the first binding member of any one of claims 57-71.

76. A method of modulating expression of an endogenous gene in a cell, the method comprising:
contacting the cell with:
(i) a polypeptide according to any one of claims 13-26 or 31-40, wherein the polypeptide NBD is fused to the first binding member; and a second binding member according to any one of claims 41-55;
(ii) a polypeptide according to any one of claims 27-40, wherein the polypeptide NBD is fused to the second binding member; and a first binding member according to any one of claims 57-71,
(iii) the composition of claim 56; or
(iv) the composition of claim 72,
wherein the polypeptide and the second binding member or the polypeptide and the first binding member penetrate the cell membrane and wherein the NBD of the polypeptide binds to a target nucleic acid sequence present in the endogenous gene and the heterologous functional domain modulates expression of the endogenous gene.

77. The method of claim 76, wherein the target nucleic acid is genomic DNA.

78. The method of claim 76 or 77, wherein the functional domain is a transcriptional activator and the target nucleic acid sequence is present in an expression control region of the gene, wherein the polypeptide increases expression of the gene.

79. The method of claim 78, wherein the transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta).

80. The method of claim 76 or 77, wherein the functional domain is a transcriptional repressor and the target nucleic acid sequence is present in an expression control region of the gene, wherein the polypeptide decreases expression of the gene.

81. The method of claim 80, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

82. The method of any of claims 76-81, wherein the gene is a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEA1 gene, a TTR gene, a NR3C1 gene, a CD52 gene, an erythroid specific enhancer of the BCL11A gene, a CELE gene, a TGFER1 gene, a SERPINA1 gene, a HEV genomic DNA in infected cells, a CEP290 gene, a DMD gene, a CFTR gene, or an IL2RG gene.

83. The method of any of claims 80-82, wherein the expression control region of the gene comprises a promoter region of the gene.

84. The method of any of claims 80-83, wherein the cell is an animal cell or plant cell.

85. The method of any of claims 80-84, wherein the cell is a human cell.

86. The method of any of claims 80-83, wherein the cell is an ex vivo cell.

87. The method of any of claims 80-86, wherein the administering comprises parenteral administration.

88. The method of any of claims 80-86, wherein the administering comprises intravenous, intramuscular, intrathecal, or subcutaneous administration.

89. The method of any of claims 80-86, wherein the administering comprises direct injection into a site in a subject.

90. The method of any of claims 80-86, wherein the administering comprises direct injection into a tumor.


SEQUENCE LISTING (186 sequences)

SEQ ID NO: 1 - protein, 34 aa, Artificial sequence
  synthetic sequence
  SITE (12)..(12): Xaa is any amino acid
  SITE (13)..(13): Xaa is any amino acid or absent
LTPDQVVAIASXXGGKQALETVQRLLPVLCQDHG

SEQ ID NO: 2 - protein, 72 aa, Artificial sequence
  synthetic sequence
DSDEHLKKLKTFLENLRRHLDRLDKHIKQLRDILSENPEDERVKDVIDLSERSVRIVKTVIKIFEDSVRKKE

SEQ ID NO: 3 - protein, 74 aa, Artificial sequence
  synthetic sequence
MDDKELDKLLDTLEKILQTATKIIDDANKLLEKLRRSERKDPKVVETYVELLKRHEKAVKELLEIAKTHAKKVE

SEQ ID NO: 4 - protein, 72 aa, Artificial sequence
  synthetic sequence
DSDEHLKKLKKFLENLRRHLDRLKKHIKQLRDILSENPEDKRVKDVIDLSERSVRIVKTVIKIFEDSVRKKE

SEQ ID NO: 5 - protein, 72 aa, Artificial sequence
  synthetic sequence
DSKEHLKKLKKFLENLRRHLDRLKKHIKQLRKILSENPEDKRVKDVIDLSERSVRIVKTVIKIFEDSVRKKE

SEQ ID NO: 6 - protein, 72 aa, Artificial sequence
  synthetic sequence
DSKKHLKKLKKFLENLRRHLDRLKKHIKQLRKILKENPEDKRVKDVIDLSERSVRIVKKVIKIFEDSVRKKE

SEQ ID NO: 7 - protein, 72 aa, Artificial sequence
  synthetic sequence
DSKKHLKKLKKFLENLRRHLDRLKKHIKQLRKILKENPEDKRVKDVIDKSERSVRIVKKVIKIFEDSVRKKE

SEQ ID NO: 8 - protein, 72 aa, Artificial sequence
  synthetic sequence
DSKKHLKKLKKFLENLRRHLDRLKKHIKQLRKILKENPKDKRVKDVIDKSERSVRIVKKVIKIFEKSVRKKE

SEQ ID NO: 9 - protein, 72 aa, Artificial sequence
  synthetic sequence
DSKKHLKKLKKFLENLRRHLDRLKKHIKQLRKILKENPKKKRVKKVIKKSERSVRIVKKVIKIFEKSVRKKE

SEQ ID NO: 10 - protein, 74 aa, Artificial sequence
  synthetic sequence
MKDKELDKLLDTLEKILQKATKIIDDANKLLEKLRRSERKKPKVVETYVELLKRHEKAVKELLEIAKTHAKKVE

SEQ ID NO: 11 - protein, 196 aa, Artificial sequence
  synthetic sequence
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 12 - protein, 75 aa, Artificial sequence
  synthetic sequence
MKDDKELDKLLDTLEKILQTATKIIDKANKLLEKLRRSKRKDPKVVETYVELLKRHEKAVKELLEIAKKHAKKVE

SEQ ID NO: 13 - protein, 74 aa, Artificial sequence
  synthetic sequence
MKDKELDKLLDKLEKILQKATKIIDKANKLLEKLRRSERKKPKVVKTYVELLKRHEKAVKELLEIAKTHAKKVE

SEQ ID NO: 14 - protein, 74 aa, Artificial sequence
  synthetic sequence
MKDKKLDKLLDKLEKILQKATKIIDKANKLLEKLRRSKRKKPKVVKTYVELLKRHEKAVKELLEIAKTHAKKVE

SEQ ID NO: 15 - protein, 75 aa, Artificial sequence
  synthetic sequence
MKKDKKLDKLLDKLEKILQKATKIIDKANKLLEKLRRSKRKKPKVVKTYVELLKRHEKAVKELLEIAKTHAKKVE

SEQ ID NO: 16 - protein, 74 aa, Artificial sequence
  synthetic sequence
MDDKKLDKLLDKLEKILQTATKIIDDANKLLEKLRRSERKDPKVVKTYVELLKRHEKAVKELLEIAKTHAKKVE

SEQ ID NO: 17 - protein, 34 aa, Artificial sequence
  synthetic sequence
  SITE (12)..(12): Xaa is any amino acid
  SITE (13)..(13): Xaa is any amino acid or absent
LTPKQVVAIASXXGGKQALETVQRLLPVLCQDHG

SEQ ID NO: 18 - protein, 34 aa, Artificial sequence
  synthetic sequence
  SITE (12)..(12): Xaa is any amino acid
  SITE (13)..(13): Xaa is any amino acid or absent
LTPRQVVAIASXXGGKQALETVQRLLPVLCQDHG

SEQ ID NO: 19 - protein, 34 aa, Artificial sequence
  synthetic sequence
  SITE (12)..(12): Xaa is any amino acid
  SITE (13)..(13): Xaa is any amino acid or absent
LTPDQVVAIAKXXGGKQALETVQRLLPVLCQDHG

SEQ ID NO: 20 - protein, 34 aa, Artificial sequence
  synthetic sequence
  SITE (12)..(12): Xaa is any amino acid
  SITE (13)..(13): Xaa is any amino acid or absent
LTPDQVVAIARXXGGKQALETVQRLLPVLCQDHG

SEQ ID NO: 21 - protein, 34 aa, Artificial sequence
  synthetic sequence
  SITE (12)..(12): Xaa is any amino acid
  SITE (13)..(13): Xaa is any amino acid or absent
LTPDQVVAIASXXGGKQALETVKRLLPVLCQDHG

SEQ ID NO: 22 - protein, 34 aa, Artificial sequence
  synthetic sequence
  SITE (12)..(12): Xaa is any amino acid
  SITE (13)..(13): Xaa is any amino acid or absent
LTPDQVVAIASXXGGKQALETVRRLLPVLCQDHG

SEQ ID NO: 23 - protein, 34 aa, Artificial sequence
  synthetic sequence
  SITE (12)..(12): Xaa is any amino acid
  SITE (13)..(13): Xaa is any amino acid or absent
LTPDQVVAIASXXGGKQALETVQRLLPVLKQDHG

SEQ ID NO: 24 - protein, 34 aa, Artificial sequence
  synthetic sequence
  SITE (12)..(12): Xaa is any amino acid
  SITE (13)..(13): Xaa is any amino acid or absent
LTPDQVVAIASXXGGKQALETVQRLLPVLRQDHG

SEQ ID NO: 25 - protein, 34 aa, Artificial sequence
  synthetic sequence
  SITE (12)..(12): Xaa is any amino acid
  SITE (13)..(13): Xaa is any amino acid or absent
LTPDQVVAIASXXGGKQALETVQRLLPVLCQKHG

SEQ ID NO: 26 - protein, 34 aa, Artificial sequence
  synthetic sequence
  SITE (12)..(12): Xaa is any amino acid
  SITE (13)..(13): Xaa is any amino acid or absent
LTPDQVVAIASXXGGKQALETVQRLLPVLCQRHG

SEQ ID NO: 27 - protein, 35 aa, Artificial sequence
  synthetic sequence
LTPEQVVAIACNKGGKQALKTVQRLLPVLCKPPYC

SEQ ID NO: 28 - protein, 35 aa, Artificial sequence
  synthetic sequence
LTPNQVVAIASNKGGKQALETVQRLLPVLCKPPHR

SEQ ID NO: 29 - protein, 35 aa, Artificial sequence
  synthetic sequence
LTPKQVVAIAGYKGANQALGTVQRLLPVLCKPPYG

SEQ ID NO: 30 - protein, 35 aa, Artificial sequence
  synthetic sequence
LTPKQVVAIANYKGAKQALETVQRLLPLLCKPPYG

SEQ ID NO: 31 - protein, 35 aa, Artificial sequence
  synthetic sequence
LTPKQVVAIASYKGANQALGTVQRLLPVLCKPPYG

SEQ ID NO: 32 - protein, 35 aa, Artificial sequence
  synthetic sequence
MTPKQVVAIASYKGANQALGTVQRLLPVLCKPPYG

SEQ ID NO: 33 - protein, 34 aa, Artificial sequence
  synthetic sequence
LTNDRLVALACIGGRSALNAVKDGLPNALTLIRR

SEQ ID NO: 34 - protein, 35 aa, Artificial sequence
  synthetic sequence
LTPAQVVAIASHNGGKQALKTVQRLLPVLCQAHGL

SEQ ID NO: 35 - protein, 34 aa, Artificial sequence
  synthetic sequence
LVTGQLLKIAKRGGVNAVEAVHASRNALTGAPLH

SEQ ID NO: 36 - protein, 35 aa, Artificial sequence
  synthetic sequence
LTPDQVVAIASNGGGKQALETVRRLLPVLCKPPYR

SEQ ID NO: 37 - protein, 35 aa, Artificial sequence
  synthetic sequence
LTPDQVVAIASNGGGKQALKTVQRLLPVLCKPPYS

SEQ ID NO: 38 - protein, 35 aa, Artificial sequence
  synthetic sequence
LTPNQVVAIASNHGGKQALETVQRLLPVLRKPPYG

SEQ ID NO: 39 - protein, 35 aa, Artificial sequence
  synthetic sequence
LTPEQVVAIASNKGGKQALETVQRLLPVLRKPPYG

SEQ ID NO: 40 - protein, 35 aa, Artificial sequence
  synthetic sequence
LLPHQVVAIVSNSGGKQALETVRRLLPVLCKPPYS

SEQ ID NO: 41 - protein, 34 aa, Artificial sequence
  synthetic sequence
LTPKQVVAIASYGGKQALETVQRLLPVLCKPPYG

SEQ ID NO: 42 - protein, 34 aa, Artificial sequence
  synthetic sequence
LTPKQVVAIASYGGKQSLETVQRLLPVLCKPPYG

SEQ ID NO: 43 - protein, 35 aa, Artificial sequence
  synthetic sequence
LTPKQVVAIASYKGANQALETVQRLLPVLCKPPYG

SEQ ID NO: 44 - protein, 34 aa, Artificial sequence
  synthetic sequence
LTNDRLVALACIGGRSALNAVKDGLPNALTLITR

SEQ ID NO: 45 - protein, 35 aa, Artificial sequence
  synthetic sequence
LTPNQVVAIASGIGGRQALETVHRLLPVLCKPPYG

SEQ ID NO: 46 - protein, 35 aa, Artificial sequence
  synthetic sequence
LTPNQVVAIASHDGGKQALETVQRLLPVLRKPPYG

SEQ ID NO: 47 - protein, 35 aa, Artificial sequence
  synthetic sequence
LTPEQVVAIASHGGAKQALKTVQRLLPVLCQNHGL

SEQ ID NO: 48 - protein, 35 aa, Artificial sequence
  synthetic sequence
LTPEQVVAIASHNGGKQALETVQRLLPVLCKPPYR

SEQ ID NO: 49 - protein, 35 aa, Artificial sequence
  synthetic sequence
LTPKQVVAIASHNGGKQALETVQRLLPVLCHPPYG

SEQ ID NO: 50 - protein, 35 aa, Artificial sequence
  synthetic sequence
LTPKQVVAIASHNGGKQALETVQRLLPVLCQPPYG

SEQ ID NO: 51 - protein, 35 aa, Artificial sequence
  synthetic sequence
LTPNQVVAIASHNGGKQALETVQRLLPVLCKPPYG

SEQ ID NO: 52 - protein, 35 aa, Artificial sequence
  synthetic sequence
LTRNQVVAIASHNGGKQALETVQRLLPVLCKEYGL

SEQ ID NO: 53 - protein, 35 aa, Artificial sequence
  synthetic sequence
LTPEQVVAIASKGGGKQALETVQRLLPVLCKPAYG

SEQ ID NO: 54 - protein, 35 aa, Artificial sequence
  synthetic sequence
LTPNQVVAIASKGGGKQALETVQRLLPVLCQPPYG

SEQ ID NO: 55 - protein, 35 aa, Artificial sequence
  synthetic sequence
LTPDQVVAIASKIGGKQALETVQRLLPVLCKPPYG

SEQ ID NO: 56 - protein, 35 aa, Artificial sequence
  synthetic sequence
LTPAQVVAIASNGGGKQALETVRRLLPVLCQAHGL

SEQ ID NO: 57 - protein, 35 aa, Artificial sequence
  synthetic sequence
LTPARVVAIASNGGGKQALQTVQRLLPVLCEQHGL

SEQ ID NO: 58 - protein, 35 aa, Artificial sequence
  synthetic sequence
LTPDQVVAIASNGGAKQALKTVQRLLPVLCQPPYG

SEQ ID NO: 59 - protein, 35 aa, Artificial sequence
  synthetic sequence
LTPNQVIAIASNGGGKQALETVQRLLPVLCKPPYG

SEQ ID NO: 60 - protein, 35 aa, Artificial sequence
  synthetic sequence
LTPNQVVAIASNHGGKQALETVQRLLPVLCKPPYN

SEQ ID NO: 61 - protein, 35 aa, Artificial sequence
  synthetic sequence
LTPAKVVAIASNIGGKQALETVQRLLPVLCQAHGL

SEQ ID NO: 62 - protein, 35 aa, Artificial sequence
  synthetic sequence
LTPAQVVAIACNIGGKQALETVRRLLPVLCQAHGL

SEQ ID NO: 63 - protein, 35 aa, Artificial sequence
  synthetic sequence
LTPAQVVAIASNIGGKQALETVQRLLPVLCRAHGL

SEQ ID NO: 64 - protein, 35 aa, Artificial sequence
  synthetic sequence
LTPAQVVAIASNIGGKQALETVRRLLPVLCQAHGL

SEQ ID NO: 65 - protein, 35 aa, Artificial sequence
  synthetic sequence
LTPDQVVAIARNIGGKQALETVRRLLPVLCQAHGL

SEQ ID NO: 66 - protein, 35 aa, Artificial sequence
  synthetic sequence
LTPDQVVAIASNIGGKQALKTVQRLLPVLCQAHGL

SEQ ID NO: 67 - protein, 35 aa, Artificial sequence
  synthetic sequence
LTPEQVVTIANNIGGKQALETVQRLLPVLRKPPYG

SEQ ID NO: 68 - protein, 35 aa, Artificial sequence
  synthetic sequence
LTPNQVVTIANNIGGKQALETVQRLLPVLCKPPYG

SEQ ID NO: 69 - protein, 35 aa, Artificial sequence
  synthetic sequence
LTPEQVVAIASNKGGKQALETVQRLLPVLCKPPYG

SEQ ID NO: 70 - protein, 35 aa, Artificial sequence
  synthetic sequence
LTPAQVVAIASNNGGKQALERVQRLLPVLCQAHGL

SEQ ID NO: 71 - protein, 35 aa, Artificial sequence
  synthetic sequence
LTPAQVVAIASNNGGKQALETVRRLLPVLCQAHGL

SEQ ID NO: 72 - protein, 35 aa, Artificial sequence
  synthetic sequence
LTPNQVVAIASNNGAKQALETVQRLLPVLCKPPHP

SEQ ID NO: 73 - protein, 35 aa, Artificial sequence
  synthetic sequence
LTPNQVVAIASNNGGKQALETVQRLLPVLCKPAYG

SEQ ID NO: 74 - protein, 35 aa, Artificial sequence
  synthetic sequence
LTPNQVVAIASNNGGKQALETVQRLLPVLCKPPHP

SEQ ID NO: 75 - protein, 35 aa, Artificial sequence
  synthetic sequence
LTREQVVAIASNNGGKQALETVQRLLPVLRQAHGL

SEQ ID NO: 76 - protein, 35 aa, Artificial sequence
  synthetic sequence
LTRNQVVAIVNNNGGKQALETVHRLLPVLCQPPHG

SEQ ID NO: 77 - protein, 35 aa, Artificial sequence
  synthetic sequence
LTRNQVVAIVNNNGGKQALETVHRLLPVLCQPPYG

SEQ ID NO: 78 - protein, 35 aa, Artificial sequence
  synthetic sequence
LTPAQVVAIASNSGGKQALETVQRLLPVLRQAHGL

SEQ ID NO: 79 - protein, 34 aa, Artificial sequence
  synthetic sequence
LSPNQVVAIASHNGGKPALETVQRLLPVLCKPPY

SEQ ID NO: 80 - protein, 34 aa, Artificial sequence
  synthetic sequence
LLPDQVVAIVSNNGGKLALGTVQRLLPVLCKPPY

SEQ ID NO: 81 - protein, 34 aa, Artificial sequence
  synthetic sequence
LTPAQVVAIASNGGKQALETVRRLLPVLCQAHGL

SEQ ID NO: 82 - protein, 34 aa, Artificial sequence
  synthetic sequence
LTPAQVVAIASNSGGKPALETVRRLLPVLCQAHG

SEQ ID NO: 83 - protein, 34 aa, Artificial sequence
  synthetic sequence
LTPDQVIAIVSNGGGKPALETVRRLLPVLCKHPY

SEQ ID NO: 84 - protein, 34 aa, Artificial sequence
  synthetic sequence
LTPDQVIAIVSNGGGKPALETVRRLLPVLCKPPY

SEQ ID NO: 85 - protein, 34 aa, Artificial sequence
  synthetic sequence
LTPDQVVTIASNNGGKPALETVRRLLPVLCKPPY

SEQ ID NO: 86 - protein, 34 aa, Artificial sequence
  synthetic sequence
LTPNQVVAIASNNGGKPALETVQRLLPVLCKPPY

SEQ ID NO: 87 - protein, 34 aa, Artificial sequence
  synthetic sequence
LTPVQVVAIASNGGKQALATVQRLLPVLCQAHGL

SEQ ID NO: 88 - protein, 34 aa, Artificial sequence
  synthetic sequence
LTPKQVVAIASYGGKQALETVQRLLPVLCQPPYG

SEQ ID NO: 89 - protein, 34 aa, Artificial sequence
  synthetic sequence
LSTTRVVSIACIGGRQALKAIKTHMPALRQAPYS

SEQ ID NO: 90 - protein, 34 aa, Artificial sequence
  synthetic sequence
LSTTRVVSIACIGGRQALEAIKTHMPALRQAPYS

SEQ ID NO: 91 - protein, 35 aa, Artificial sequence
  synthetic sequence
LTPQQVVAIASNTGGKQALEAVTVQLRVLRGARYG

SEQ ID NO: 92 - protein, 35 aa, Artificial sequence
  synthetic sequence
LTPQQVVAIASNTGGKRALEAVCVQLPVLRAAPYR

SEQ ID NO: 93 - protein, 35 aa, Artificial sequence
  synthetic sequence
LSTAQVVAVAGRNGGKQALEAVRAQLPALRAAPYG

SEQ ID NO: 94 - protein, 35 aa, Artificial sequence
  synthetic sequence
LSIAQVVAVASRSGGKQALEAVRAQLLALRAAPYG

SEQ ID NO: 95 - protein, 34 aa, Artificial sequence
  synthetic sequence
LSTAQVVAVASGSGGKPALEAVRAQLLALRAAPY

SEQ ID NO: 96 - protein, 35 aa, Artificial sequence
  synthetic sequence
LSTAQVVAVASGSGGKQALEAVRVQLLALRAAPYG

SEQ ID NO: 97 - protein, 35 aa, Artificial sequence
  synthetic sequence
LSTAQVVAVASGSGGKPALEAVRAQLLALRAAPYG

SEQ ID NO: 98 - protein, 35 aa, Artificial sequence; identical to SEQ ID NO: 97
  synthetic sequence

SEQ ID NO: 99 - protein, 35 aa, Artificial sequence
  synthetic sequence
LNTAQVVAIASHDGGKPALEAVRAKLPVLRGVPYA

SEQ ID NO: 100 - protein, 35 aa, Artificial sequence
  synthetic sequence
LSTAQVVAVASHDGGKPALEAVRKQLPVLRGVPHQ

SEQ ID NO: 101 - protein, 35 aa, Artificial sequence; identical to SEQ ID NO: 100
  synthetic sequence

SEQ ID NO: 102 - protein, 35 aa, Artificial sequence
  synthetic sequence
LSTEQVVAIASHNGGKQALEAVKAQLPVLRRAPYG

SEQ ID NO: 103 - protein, 35 aa, Artificial sequence
  synthetic sequence
LSVAQVVTIASHNGGKQALEAVRAQLLALRAAPYG

SEQ ID NO: 104 - protein, 35 aa, Artificial sequence
  synthetic sequence
LNTAQVVAIASHYGGKPALEAVWAKLPVLRGVPYA

SEQ ID NO: 105 - protein, 35 aa, Artificial sequence
  synthetic sequence
LSTAQVVAIASNGGGKQALEGIGEQLRKLRTAPYG

SEQ ID NO: 106 - protein, 35 aa, Artificial sequence
  synthetic sequence
LSPEQVVAIASNHGGKQALEAVRALFRGLRAAPYG

SEQ ID NO: 107 - protein, 35 aa, Artificial sequence
  synthetic sequence
LSTEQVVAIASNHGGKQALEAVRALFRGLRAAPYG

SEQ ID NO: 108 - protein, 35 aa, Artificial sequence
  synthetic sequence
LSTEQVVAIASNKGGKQALEAVKAQLLALRAAPYA

SEQ ID NO: 109 - protein, 35 aa, Artificial sequence
  synthetic sequence
LSTEQVVAIASNNGGKQALEAVKAQLPVLRRAPCG

SEQ ID NO: 110 - protein, 35 aa, Artificial sequence
  synthetic sequence
LSTEQVVAIASNNGGKQALEAVKAQLPVLRRAPYG

SEQ ID NO: 111 - protein, 35 aa, Artificial sequence
  synthetic sequence
LSTEQVVAVASNNGGKQALKAVKAQLLALRAAPYE

SEQ ID NO: 112 - protein, 35 aa, Artificial sequence
  synthetic sequence
LSTAQLVAIASNPGGKQALEAIRALFRELRAAPYA

SEQ ID NO: 113 - protein, 35 aa, Artificial sequence
  synthetic sequence
LSTAQLVAIASNPGGKQALEAVRALFRELRAAPYA

SEQ ID NO: 114 - protein, 35 aa, Artificial sequence
  synthetic sequence
LSTAQLVAIASNPGGKQALEAVRAPFREVRAAPYA

SEQ ID NO: 115 - protein, 35 aa, Artificial sequence
  synthetic sequence
LSTAQLVSIASNPGGKQALEAVRALFRELRAAPYA

SEQ ID NO: 116 - protein, 35 aa, Artificial sequence
  synthetic sequence
LSTAQVVAIASNPGGKQALEAVRALFRELRAAPYA

SEQ ID NO: 117 - protein, 35 aa, Artificial sequence
  synthetic sequence
LTPQQVVAIASNTGGKRALEAVRVQLPVLRAAPYE

SEQ ID NO: 118 - protein, 35 aa, Artificial sequence
  synthetic sequence
LSTAQVVAIATRSGGKQALEAVRAQLLDLRAAPYG

SEQ ID NO: 119 - protein, 35 aa, Artificial sequence
  synthetic sequence
LSTAQVVAIASSHGGKQALEAVRALFRELRAAPYG

SEQ ID NO: 120 - protein, 35 aa, Artificial sequence
  synthetic sequence
LSTAQVATIASSIGGRQALEALKVQLPVLRAAPYG

SEQ ID NO: 121 - protein, 35 aa, Artificial sequence
  synthetic sequence
LSTAQVATIASSIGGRQALEAVKVQLPVLRAAPYG

SEQ ID NO: 122 - protein, 33 aa, Artificial sequence
  synthetic sequence
FRQADIVKIASNGGSAQALNAVIKLGPTLRQRG

SEQ ID NO: 123 - protein, 33 aa, Artificial sequence
  synthetic sequence
FRQADIVKMASNGGSAQALNAVIKLGPTLRQRG

SEQ ID NO: 124 - protein, 33 aa, Artificial sequence
  synthetic sequence
FRQTDIVKMAGSGGSAQALNAVIKHGPTLRQRG

SEQ ID NO: 125 - protein, 33 aa, Artificial sequence
  synthetic sequence
FNRADIVRIAGNGGGAQALYSVRDAGPTLGKRG

SEQ ID NO: 126 - protein, 33 aa, Artificial sequence
  synthetic sequence
FSRADIVRIAGNGGGAQALYSVLDVGPTLGKRG

SEQ ID NO: 127 - protein, 33 aa, Artificial sequence
  synthetic sequence
LQRADIVKIAGNGGGAQALQAVITHRAALTQAG

SEQ ID NO: 128 - protein, 33 aa, Artificial sequence
  synthetic sequence
FSATDIVKIASNIGGAQALQAVISRRAALIQAG

SEQ ID NO: 129 - protein, 33 aa, Artificial sequence
  synthetic sequence
FSAADIVKIASNNGGAQALQAVISRRAALIQAG

SEQ ID NO: 130 - protein, 33 aa, Artificial sequence
  synthetic sequence
FTLTDIVKMAGNNGGAQALKVVLEHGPTLRQRG

SEQ ID NO: 131 - protein, 33 aa, Artificial sequence
  synthetic sequence
FNTEQIVRMVSHDGGSLNLKAVKKYHDALRERK

SEQ ID NO: 132 - protein, 33 aa, Artificial sequence
  synthetic sequence
LDRQQILRIASHDGGSKNIAAVQKFLPKLMNFG

SEQ ID NO: 133 - protein, 33 aa, Artificial sequence
  synthetic sequence
FSAKHIVRIAAHIGGSLNIKAVQQAQQALKELG

SEQ ID NO: 134 - protein, 33 aa, Artificial sequence
  synthetic sequence
LGHKELIKIAARNGGGNNLIAVLSCYAKLKEMG

SEQ ID NO: 135 - protein, 33 aa, Artificial sequence
  synthetic sequence
FNAEQIVRMVSHKGGSKNLALVKEYFPVFSSFH

SEQ ID NO: 136 - protein, 33 aa, Artificial sequence; identical to SEQ ID NO: 135
  synthetic sequence

SEQ ID NO: 137 - protein, 33 aa, Artificial sequence
  synthetic sequence
FNAEQIVSMVSNGGGSLNLKAVKKYHDALKDRG

SEQ ID NO: 138 - protein, 33 aa, Artificial sequence
  synthetic sequence
LEPKDIVSIASHIGATQAITTLLNKWAALRAKG

SEQ ID NO: 139 - protein, 33 aa, Artificial sequence
  synthetic sequence
FNRASIVKIAGNSGGAQALQAVLKHGPTLDERG

SEQ ID NO: 140 - protein, 11 aa, Artificial sequence
  synthetic sequence
GKGSKGKGKGK

SEQ ID NO: 141 - protein, 14 aa, Artificial sequence
  synthetic sequence
GKGSKGKGKGKGSK

SEQ ID NO: 142 - protein, 153 aa, Artificial sequence
  synthetic sequence
MKDKELDKLLDTLEKILQKATKIIDDANKLLEKLRRSERKKPKVVETYVELLKRHEKAVKELLEIAKTHAKKVEGSGGGGGMDAKSLTAWSRTLVTFKDVFVDFTREEWKLLDTAQQIVYRNVMLENYKNLVSLGYQLTKPDVILRLEKGEEP

SEQ ID NO: 143 - protein, 153 aa, Artificial sequence
  synthetic sequence
MDDKKLDKLLDKLEKILQTATKIIDDANKLLEKLRRSERKDPKVVKTYVELLKRHEKAVKELLEIAKTHAKKVEGSGGGGGMDAKSLTAWSRTLVTFKDVFVDFTREEWKLLDTAQQIVYRNVMLENYKNLVSLGYQLTKPDVILRLEKGEEP

SEQ ID NO: 144 - protein, 154 aa, Artificial sequence
  synthetic sequence
MKDDKELDKLLDTLEKILQTATKIIDKANKLLEKLRRSKRKDPKVVETYVELLKRHEKAVKELLEIAKKHAKKVEGSGGGGGMDAKSLTAWSRTLVTFKDVFVDFTREEWKLLDTAQQIVYRNVMLENYKNLVSLGYQLTKPDVILRLEKGEEP

SEQ ID NO: 145 - protein, 153 aa, Artificial sequence
  synthetic sequence
MKDKELDKLLDKLEKILQKATKIIDKANKLLEKLRRSERKKPKVVKTYVELLKRHEKAVKELLEIAKTHAKKVEGSGGGGGMDAKSLTAWSRTLVTFKDVFVDFTREEWKLLDTAQQIVYRNVMLENYKNLVSLGYQLTKPDVILRLEKGEEP

SEQ ID NO: 146 - protein, 153 aa, Artificial sequence
  synthetic sequence
MKDKKLDKLLDKLEKILQKATKIIDKANKLLEKLRRSKRKKPKVVKTYVELLKRHEKAVKELLEIAKTHAKKVEGSGGGGGMDAKSLTAWSRTLVTFKDVFVDFTREEWKLLDTAQQIVYRNVMLENYKNLVSLGYQLTKPDVILRLEKGEEP

SEQ ID NO: 147 - protein, 158 aa, Artificial sequence
  synthetic sequence
MKKDKKLDKLLDKLEKILQKATKIIDKANKLLEKLRRSKRKKPKVVKTYVELLKRHEKAVKELLEIAKTHAKKVEGKGSKGKGKGKMDAKSLTAWSRTLVTFKDVFVDFTREEWKLLDTAQQIVYRNVMLENYKNLVSLGYQLTKPDVILRLEKGEEP

SEQ ID NO: 148 - protein, 75 aa, Artificial sequence
  synthetic sequence
PTDEVIEVLKELLRIHRENLRVNEEIVEVNERASRVTDREELERLLRRSNELIKRSRELNEESKKLIEKLERLAT

SEQ ID NO: 149 - protein, 74 aa, Artificial sequence
  synthetic sequence
DNEEIIKEARRVVEEYKKAVDRLEELVRRAENAKHASEKELKDIVREILRISKELNKVSERLIELWERSQERAR

SEQ ID NO: 150 - protein, 72 aa, Artificial sequence
  synthetic sequence
TAEELLEVHKKSDRVTKEHLRVSEEILKVVEVLTRGEVSSEVLKRVLRKLEELTDKLRRVTEEQRRVVEKLN

SEQ ID NO: 151 - protein, 76 aa, Artificial sequence
  synthetic sequence
DLEDLLRRLRRLVDEQRRLVEELERVSRRLEKAVRDNEDERELARLSREHSDIQDKHDKLAREILEVLKRLLERTE

SEQ ID NO: 152 - protein, 72 aa, Artificial sequence
  synthetic sequence
PEDDVVRIIKEDLESNREVLREQKEIHRILELVTRGEVSEEAIDRVLKRQEDLLKKQKESTDKARKVVEERR

SEQ ID NO: 153 - protein, 70 aa, Artificial sequence
  synthetic sequence
DEVRLITEWLKLSEESTRLLKELVELTRLLRNNVPNVEEILREHERISRELERLSRRLKDLADKLERTRR

SEQ ID NO: 154 - protein, 72 aa, Artificial sequence
  synthetic sequence
DEEDHLKKLKTHLEKLERHLKLLEDHAKKLEDILKERPEDSAVKESIDELRRSIELVRESIEIFRQSVEEEE

SEQ ID NO: 155 - protein, 74 aa, Artificial sequence
  synthetic sequence
GDVKELTKILDTLTKILETATKVIKDATKLLEEHRKSDKPDPRLIETHKKLVEEHETLVRQHKELAEEHLKRTR

SEQ ID NO: 156 - protein, 75 aa, Artificial sequence; identical to SEQ ID NO: 15
  synthetic sequence

SEQ ID NO: 157 - protein, 75 aa, Artificial sequence; identical to SEQ ID NO: 15
  synthetic sequence

SEQ ID NO: 158 - protein, 34 aa, Artificial sequence
  synthetic sequence
  SITE (12)..(12): Xaa is any amino acid
  SITE (13)..(13): Xaa is any amino acid or absent
  SITE (20)..(20): Xaa is Arg, Lys or His
LTPDQVVAIASXXGGKQALXTVQRLLPVLCQDHG

SEQ ID NO: 159 - protein, 34 aa, Artificial sequence
  synthetic sequence
  SITE (12)..(12): Xaa is any amino acid
  SITE (13)..(13): Xaa is any amino acid or absent
  SITE (31)..(31): Xaa is Lys, Arg, or His
LTPDQVVAIASXXGGKQALETVQRLLPVLCXDHG

SEQ ID NO: 160 - protein, 34 aa, Artificial sequence
  synthetic sequence
  SITE (12)..(12): Xaa is any amino acid
  SITE (13)..(13): Xaa is any amino acid or absent
  SITE (20)..(20): Xaa is Lys, Arg or His
  SITE (31)..(31): Xaa is Lys, Arg or His
LTPDQVVAIASXXGGKQALXTVQRLLPVLCXDHG

SEQ ID NO: 161 - protein, 34 aa, Artificial sequence
  synthetic sequence
  SITE (12)..(12): Xaa is any amino acid
  SITE (13)..(13): Xaa is any amino acid or absent
LTPDQVVAIASXXGGKQALRTVQRLLPVLCKDHG

SEQ ID NO: 162 - protein, 21 aa, Artificial sequence
  synthetic sequence
GKGSKGKGKGKMDAKSLTAWS

SEQ ID NO: 163 - protein, 15 aa, Artificial sequence
  synthetic sequence
GGGGGMDAKSLTAWS

SEQ ID NO: 164 - protein, 13 aa, Artificial sequence
  synthetic sequence
GGGMDAKSLTAWS

SEQ ID NO: 165 - protein, 9 aa, Artificial sequence
  synthetic sequence
GSKGKGKGK

SEQ ID NO: 166 - protein, 19 aa, Artificial sequence
  synthetic sequence
GSKGKGKGKMDAKSLTAWS

SEQ ID NO: 167 - protein, 21 aa, Artificial sequence
  synthetic sequence
GGGSKGKGKGKMDAKSLTAWS

SEQ ID NO: 168 - protein, 1117 aa, Artificial sequence
  synthetic sequence
MAHHHHHHLATTHMGSSNSNNATMAPDRVRAVSHWSSGGSMASMTGGQQMGGGAGKPIPNPLLGLDSTGAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNHGGKQALETVQRLLPVLCKDHGLTPDQVVAIASNHGGKQALETVQRLLPVLCKDHGLTPDQVVAIASHDGGKQALETVQRLLPVLCKDHGLTPDQVVAIASNIGGKQALETVQRLLPVLCKDHGLTPDQVVAIASNHGGKQALETVQRLLPVLCKDHGLTPDQVVAIASNGGGKQALETVQRLLPVLCKDHGLTPDQVVAIASNHGGKQALETVQRLLPVLCKDHGLTPDQVVAIASNGGGKQALETVQRLLPVLCKDHGLTPDQVVAIASNGGGKQALETVQRLLPVLCKDHGLTPDQVVAIASNIGGKQALETVQRLLPVLCKDHGLTPDQVVAIASHDGGKQALETVQRLLPVLCKDHGLTPDQVVAIASNGGGKQALETVQRLLPVLCKDHGLTPDQVVAIASNIGGKQALETVQRLLPVLCKDHGLTPDQVVAIASNGGGKQALETVQRLLPVLCKDHGLTPDQVVAIASNIGGKQALETVQRLLPVLCKDHGLTPEQVVAIASNIGGRPALESIVAQLSRPDPALAALTNDHLVALACLGGRPALDAVKKGLPHAPALIKRTNRRIPERTSHRVAGSGGGMDAKSLTAWSGKGSKGKGKGKGSKDSKKHLKKLKKFLENLRRHLDRLKKHIKQLRKILKENPKDKRVKDVIDKSERSVRIVKKVIKIFEKSVRKKEGGGGGMDAKSLTAWSGKGSKGKGKGKGSKDSKKHLKKLKKFLENLRRHLDRLKKHIKQLRKILKENPKDKRVKDVIDKSERSVRIVKKVIKIFEKSVRKKEGGGGGMDAKSLTAWSGKGSKGKGKGKGSKDSKKHLKKLKKFLENLRRHLDRLKKHIKQLRKILKENPKDKRVKDVIDKSERSVRIVKKVIKIFEKSVRKKE

SEQ ID NO: 169 - protein, 6 aa, Artificial sequence
  synthetic sequence
HHHHHH

SEQ ID NO: 170 - protein, 12 aa, Artificial sequence
  synthetic sequence
PDRVRAVSHWSS

SEQ ID NO: 171 - protein, 11 aa, Artificial sequence
  synthetic sequence
MASMTGGQQMG

SEQ ID NO: 172 - protein, 14 aa, Artificial sequence
  synthetic sequence
GKPIPNPLLGLDST

SEQ ID NO: 173 - protein, 7 aa, Artificial sequence
  synthetic sequence
PKKKRKV

SEQ ID NO: 174 - protein, 86 aa, Artificial sequence
  synthetic sequence
GKGSKGKGKGKGSKDSKKHLKKLKKFLENLRRHLDRLKKHIKQLRKILKENPKDKRVKDVIDKSERSVRIVKKVIKIFEKSVRKKE

SEQ ID NO: 175 - protein, 1135 aa, Artificial sequence
  synthetic sequence
MAHHHHHHLATTHMGSSNSNNATMAPDRVRAVSHWSSGGSMASMTGGQQMGGGAGKPIPNPLLGLDSTGAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNHGGKQALRTVQRLLPVLCKDHGLTPDQVVAIASNHGGKQALRTVQRLLPVLCKDHGLTPDQVVAIASHDGGKQALRTVQRLLPVLCKDHGLTPDQVVAIASNIGGKQALRTVQRLLPVLCKDHGLTPDQVVAIASNHGGKQALRTVQRLLPVLCKDHGLTPDQVVAIASNGGGKQALRTVQRLLPVLCKDHGLTPDQVVAIASNHGGKQALRTVQRLLPVLCKDHGLTPDQVVAIASNGGGKQALRTVQRLLPVLCKDHGLTPDQVVAIASNGGGKQALRTVQRLLPVLCKDHGLTPDQVVAIASNIGGKQALRTVQRLLPVLCKDHGLTPDQVVAIASHDGGKQALRTVQRLLPVLCKDHGLTPDQVVAIASNGGGKQALRTVQRLLPVLCKDHGLTPDQVVAIASNIGGKQALRTVQRLLPVLCKDHGLTPDQVVAIASNGGGKQALRTVQRLLPVLCKDHGLTPDQVVAIASNIGGKQALRTVQRLLPVLCKDHGLTPEQVVAIASNIGGRPALESIVAQLSRPDPALAALTNDHLVALACLGGRPALDAVKKGLPHAPALIKRTNRRIPERTSHRVAGSGSKGKGKGKMDAKSLTAWSGKGSKGKGKGKGSKDSKKHLKKLKKFLENLRRHLDRLKKHIKQLRKILKENPKDKRVKDVIDKSERSVRIVKKVIKIFEKSVRKKEGGGSKGKGKGKMDAKSLTAWSGKGSKGKGKGKGSKDSKKHLKKLKKFLENLRRHLDRLKKHIKQLRKILKENPKDKRVKDVIDKSERSVRIVKKVIKIFEKSVRKKEGGGSKGKGKGKMDAKSLTAWSGKGSKGKGKGKGSKDSKKHLKKLKKFLENLRRHLDRLKKHIKQLRKILKENPKDKRVKDVIDKSERSVRIVKKVIKIFEKSVRKKE

SEQ ID NO: 176 - protein, 428 aa, Artificial sequence
  synthetic sequence
MATTHNHHHHHHHHHPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPREPTTLEVLFQGPDAYPYDVPDYAGAPKKKRKVGAKKDKKLDKLLDKLEKILQKATKIIDKANKLLEKLRRSKRKKPKVVKTYVELLKRHEKAVKELLEIAKTHAKKVEGKGSKGKGKGKMDAKSLTAWSRTLVTFKDVFVDFTREEWKLLDTAQQIVYRNVMLENYKNLVSLGYQLTKPDVILRLEKGEEP

SEQ ID NO: 177 - protein, 9 aa, Artificial sequence
  synthetic sequence
HHHHHHHHH

SEQ ID NO: 178 - protein, 216 aa, Artificial sequence
  synthetic sequence
PILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPK

SEQ ID NO: 179 - protein, 8 aa, Artificial sequence
  synthetic sequence
LEVLFQGP

SEQ ID NO: 180 - protein, 7 aa, Artificial sequence
  synthetic sequence
YDVPDYA

SEQ ID NO: 181 - protein, 74 aa, Artificial sequence
  synthetic sequence
KKDKKLDKLLDKLEKILQKATKIIDKANKLLEKLRRSKRKKPKVVKTYVELLKRHEKAVKELLEIAKTHAKKVE

SEQ ID NO: 182 - protein, 72 aa, Artificial sequence
  synthetic sequence
MDAKSLTAWSRTLVTFKDVFVDFTREEWKLLDTAQQIVYRNVMLENYKNLVSLGYQLTKPDVILRLEKGEEP

SEQ ID NO: 183 - protein, 34 aa, Artificial sequence
  synthetic sequence
  SITE (12)..(12): Xaa is any amino acid
  SITE (13)..(13): Xaa is any amino acid or absent
LTPDQVVAIASXXGGKQALETVQRLLPVLCKDHG

SEQ ID NO: 184 - protein, 144 aa, Artificial sequence
  synthetic sequence
GIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 185 - protein, 65 aa, Artificial sequence
  synthetic sequence
SIVAQLSRPDPALAALTNDHLVALACLGGRPALDAVKKGLPHAPALIKRTNRRIPERTSHRVAGS

SEQ ID NO: 186 - protein, 20 aa, Artificial sequence
  synthetic sequence
  SITE (12)..(12): Xaa is any amino acid
  SITE (13)..(13): Xaa is any amino acid or absent
LTPEQVVAIASXXGGRPALE